SEQUENCE LISTING (24 sequences)

SEQ ID NO: 1 - DNA, 5986 bp, Artificial Sequence
  Production plasmid 1
  repeat_region (1)..(130): ITR
  promoter (213)..(678): hSynapsin
  misc_feature (690)..(695): Kozak
  CDS (696)..(2195): human MeCP2alpha
  misc_feature (2190)..(2195): Stop codons
  polyA_signal (2303)..(2534): SV40 late polyadenylation
  repeat_region (2599)..(2728): ITR
  misc_feature (2905)..(3343): f1 ori
  misc_feature (3372)..(4014): pUC origin of replication
  misc_feature (4689)..(5504): Kan-r
  misc_feature (5747)..(5932): Lac promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggctgctgcagctgctgctgcaccttctggtggtggcggaggcggagaggaagagagactggaagagaagtccgaggaccaggatctgcagggcctgaaggacaagcccctgaagttcaagaaagtgaagaaggataagaaagaggaaaaagagggcaagcacgagcccgtgcagccatctgctcaccattctgccgaacctgccgaagccggaaaggccgaaacatctgaaggctctggaagcgcccctgccgtgcctgaagcttctgcttctcctaagcagcggcggagcatcatcagagacagaggccctatgtacgacgaccccacactgcctgaaggctggaccagaaagctgaagcagagaaagagcggcagaagcgccgggaagtacgacgtgtacctgatcaaccctcagggcaaagccttccggtccaaggtggaactgatcgcctacttcgagaaagtgggcgacacctctctggaccccaacgacttcgatttcaccgtgacaggcagaggcagccccagcagaagagagcagaagcctcctaagaagcctaagagccccaaggctcctggcaccggaagaggtagaggtagacctaaaggcagcggcaccacaagacctaaggccgctacttctgagggcgtgcaagtgaagagagtgctggaaaagagccccggcaagctgctggtcaagatgccctttcagacaagccctggcggcaaagcagaaggcggaggggctacaacaagcacccaagtgatggtcatcaagaggcccggcagaaagcggaaggccgaagctgatcctcaggctatccccaagaagagaggccggaagcctggatctgtggttgctgctgccgccgcagaggccaagaaaaaggccgtgaaagagtccagcatccgcagcgtgcaagagacagtgctgcccatcaagaagcgcaagacccgggaaaccgtgtccatcgaagtgaaagaggtggtcaagcctctgctggtgtctaccctgggcgagaagtctggcaagggactgaaaacctgcaagtcccctggcagaaagagcaaagagtctagccctaagggcagaagcagcagcgctagctccccacctaagaaagaacaccaccatcaccaccaccatagcgagtccccaaaggctccagttcctctgcttcctccactgcctccacctccacctgagcctgagtctagcgaggatcctacaagccctcctgagccacaggatctgagcagctccgtgtgcaaagaagagaagatgcccagaggcggctccctggaatctgacggctgtcctaaagagcccgccaagacacagcctgccgttgccacagctgcaacagctgccgagaagtacaagcacagaggcgagggcgagcgcaaggatatcgtgtctagcagcatgcccagacctaaccgcgaggaacccgtggatagcagaacccctgtgaccgagagggtgtcctgatgactcgacggcgcgcccatggcgatatccaattggagctcgcggccgcctcgacttaccggtttgatatcttctagaaggatccaacgcgtaggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacaccatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 2 - protein, 498 aa, Artificial Sequence [sequence omitted]

SEQ ID NO: 3 - DNA, 1500 bp, Artificial Sequence
  human MeCP2alpha
atggctgctgcagctgctgctgcaccttctggtggtggcggaggcggagaggaagagagactggaagagaagtccgaggaccaggatctgcagggcctgaaggacaagcccctgaagttcaagaaagtgaagaaggataagaaagaggaaaaagagggcaagcacgagcccgtgcagccatctgctcaccattctgccgaacctgccgaagccggaaaggccgaaacatctgaaggctctggaagcgcccctgccgtgcctgaagcttctgcttctcctaagcagcggcggagcatcatcagagacagaggccctatgtacgacgaccccacactgcctgaaggctggaccagaaagctgaagcagagaaagagcggcagaagcgccgggaagtacgacgtgtacctgatcaaccctcagggcaaagccttccggtccaaggtggaactgatcgcctacttcgagaaagtgggcgacacctctctggaccccaacgacttcgatttcaccgtgacaggcagaggcagccccagcagaagagagcagaagcctcctaagaagcctaagagccccaaggctcctggcaccggaagaggtagaggtagacctaaaggcagcggcaccacaagacctaaggccgctacttctgagggcgtgcaagtgaagagagtgctggaaaagagccccggcaagctgctggtcaagatgccctttcagacaagccctggcggcaaagcagaaggcggaggggctacaacaagcacccaagtgatggtcatcaagaggcccggcagaaagcggaaggccgaagctgatcctcaggctatccccaagaagagaggccggaagcctggatctgtggttgctgctgccgccgcagaggccaagaaaaaggccgtgaaagagtccagcatccgcagcgtgcaagagacagtgctgcccatcaagaagcgcaagacccgggaaaccgtgtccatcgaagtgaaagaggtggtcaagcctctgctggtgtctaccctgggcgagaagtctggcaagggactgaaaacctgcaagtcccctggcagaaagagcaaagagtctagccctaagggcagaagcagcagcgctagctccccacctaagaaagaacaccaccatcaccaccaccatagcgagtccccaaaggctccagttcctctgcttcctccactgcctccacctccacctgagcctgagtctagcgaggatcctacaagccctcctgagccacaggatctgagcagctccgtgtgcaaagaagagaagatgcccagaggcggctccctggaatctgacggctgtcctaaagagcccgccaagacacagcctgccgttgccacagctgcaacagctgccgagaagtacaagcacagaggcgagggcgagcgcaaggatatcgtgtctagcagcatgcccagacctaaccgcgaggaacccgtggatagcagaacccctgtgaccgagagggtgtcctgatga

SEQ ID NO: 4 - DNA, 6769 bp, Artificial Sequence
  Production plasmid 2
  repeat_region (1)..(130): 5′ ITR
  promoter (198)..(579): CMV IE promoter
  promoter (582)..(863): CB promoter
  TATA_signal (836)..(839): TATA
  Intron (957)..(1929): chicken beta-actin intron
  misc_feature (1993)..(3486): MECP2 e1
  misc_feature (3490)..(3537): SpA
  polyA_signal (3604)..(3730): Rabbit globin poly A
  repeat_region (3819)..(3948): 3′ ITR
  rep_origin (4125)..(4580): fi ori
  misc_feature (4711)..(5568): AP(R)
  rep_origin (5742)..(6330): Origin of replication
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctgtacggaagtgttacttctgctctaaaagctgcggaattgtacccgcgggcccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctagaataaagagctcagatgcatcgatcagagtgtgttggttttttgtgtgacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 5 - DNA, 1494 bp, Artificial Sequence
  MECP2
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagc

SEQ ID NO: 6 - DNA, 6060 bp, Artificial Sequence
  Production plasmid 3
  repeat_region (1)..(130): ITR
  promoter (213)..(678): hSynapsin promoter
  misc_feature (690)..(695): Kozak
  misc_feature (696)..(2195): human MeCP2 coding sequence
  misc_feature (2190)..(2195): stop codons
  misc_feature (2256)..(2277): miRNA183
  misc_feature (2282)..(2303): miRNA183
  misc_feature (2310)..(2331): miRNA183
  misc_feature (2338)..(2359): miRNA183
  polyA_signal (2377)..(2608): SV40 late polyadenylation signal
  repeat_region (2673)..(2802): ITR
  misc_feature (2979)..(3417): f1 ori
  misc_feature (3446)..(4088): pUC origin of replication
  misc_feature (4763)..(5578): Kan-r
  misc_feature (5821)..(6006): Lac promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggctgctgcagctgctgctgcaccttctggtggtggcggaggcggagaggaagagagactggaagagaagtccgaggaccaggatctgcagggcctgaaggacaagcccctgaagttcaagaaagtgaagaaggataagaaagaggaaaaagagggcaagcacgagcccgtgcagccatctgctcaccattctgccgaacctgccgaagccggaaaggccgaaacatctgaaggctctggaagcgcccctgccgtgcctgaagcttctgcttctcctaagcagcggcggagcatcatcagagacagaggccctatgtacgacgaccccacactgcctgaaggctggaccagaaagctgaagcagagaaagagcggcagaagcgccgggaagtacgacgtgtacctgatcaaccctcagggcaaagccttccggtccaaggtggaactgatcgcctacttcgagaaagtgggcgacacctctctggaccccaacgacttcgatttcaccgtgacaggcagaggcagccccagcagaagagagcagaagcctcctaagaagcctaagagccccaaggctcctggcaccggaagaggtagaggtagacctaaaggcagcggcaccacaagacctaaggccgctacttctgagggcgtgcaagtgaagagagtgctggaaaagagccccggcaagctgctggtcaagatgccctttcagacaagccctggcggcaaagcagaaggcggaggggctacaacaagcacccaagtgatggtcatcaagaggcccggcagaaagcggaaggccgaagctgatcctcaggctatccccaagaagagaggccggaagcctggatctgtggttgctgctgccgccgcagaggccaagaaaaaggccgtgaaagagtccagcatccgcagcgtgcaagagacagtgctgcccatcaagaagcgcaagacccgggaaaccgtgtccatcgaagtgaaagaggtggtcaagcctctgctggtgtctaccctgggcgagaagtctggcaagggactgaaaacctgcaagtcccctggcagaaagagcaaagagtctagccctaagggcagaagcagcagcgctagctccccacctaagaaagaacaccaccatcaccaccaccatagcgagtccccaaaggctccagttcctctgcttcctccactgcctccacctccacctgagcctgagtctagcgaggatcctacaagccctcctgagccacaggatctgagcagctccgtgtgcaaagaagagaagatgcccagaggcggctccctggaatctgacggctgtcctaaagagcccgccaagacacagcctgccgttgccacagctgcaacagctgccgagaagtacaagcacagaggcgagggcgagcgcaaggatatcgtgtctagcagcatgcccagacctaaccgcgaggaacccgtggatagcagaacccctgtgaccgagagggtgtcctgatgactcgacggcgcgcccatggcgatatccaattggagctcgcggccgcctcgacttaccggtagtgaattctaccagtgccataggatagtgaattctaccagtgccatacacgtgagtgaattctaccagtgccatagcatgcagtgaattctaccagtgccataggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgatcctccggcgttcagcctgtgccacagccgacaggatggtgaccaccatttgccccatatcaccgtcggtactgatcccgtcgtcaataaaccgaaccgctacaccctgagcatcaaactcttttatcagttggatcatgtcggcggtgtcgcggccaagacggtcgagcttcttcaccagaatgacatcaccttcctccaccttcatcctcagcaaatccagcccttcccgatctgttgaactgccggatgccttgtcggtaaagatgcggttagcttttacccctgcatctttgagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacaccatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 7 - DNA, 22 bp, Artificial Sequence
  miR183 binding site
agtgaattctaccagtgccata

SEQ ID NO: 8 - protein, 486 aa, Artificial Sequence
  hMECP2 Isoform A
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 9 - DNA, 23 bp, Artificial Sequence
  miRNA target sequence.
agcaaaaatgtgctagtgccaaa

SEQ ID NO: 10 - DNA, 24 bp, Artificial Sequence
  miRNA target sequence.
agtgtgagttctaccattgccaaa

SEQ ID NO: 11 - DNA, 23 bp, Artificial Sequence
  miRNA target sequence.
agggattcctgggaaaactggac

SEQ ID NO: 12 - DNA, 666 bp, Artificial Sequence
  chicken beta actin promoter with a cytomegalovirus enhancer (CB7)
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 13 - DNA, 1180 bp, Artificial Sequence
  human elongation initiation factor 1 alpha promoter (EF1a)
tggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 16 bp, Artificial Sequence
  spacer
atgacttaaaccaggt

SEQ ID NO: 15 - DNA, 2802 bp, Artificial Sequence
  expression vector
  repeat_region (1)..(130): 5′ITR
  promoter (213)..(678): hSynapsin
  misc_feature (690)..(695): Kozak
  misc_feature (696)..(2195): human Mecp2alpha
  misc_binding (2256)..(2359): (4x)
  polyA_signal (2377)..(2608): SV40 late polyadenylation signal
  repeat_region (2673)..(2802): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggctgctgcagctgctgctgcaccttctggtggtggcggaggcggagaggaagagagactggaagagaagtccgaggaccaggatctgcagggcctgaaggacaagcccctgaagttcaagaaagtgaagaaggataagaaagaggaaaaagagggcaagcacgagcccgtgcagccatctgctcaccattctgccgaacctgccgaagccggaaaggccgaaacatctgaaggctctggaagcgcccctgccgtgcctgaagcttctgcttctcctaagcagcggcggagcatcatcagagacagaggccctatgtacgacgaccccacactgcctgaaggctggaccagaaagctgaagcagagaaagagcggcagaagcgccgggaagtacgacgtgtacctgatcaaccctcagggcaaagccttccggtccaaggtggaactgatcgcctacttcgagaaagtgggcgacacctctctggaccccaacgacttcgatttcaccgtgacaggcagaggcagccccagcagaagagagcagaagcctcctaagaagcctaagagccccaaggctcctggcaccggaagaggtagaggtagacctaaaggcagcggcaccacaagacctaaggccgctacttctgagggcgtgcaagtgaagagagtgctggaaaagagccccggcaagctgctggtcaagatgccctttcagacaagccctggcggcaaagcagaaggcggaggggctacaacaagcacccaagtgatggtcatcaagaggcccggcagaaagcggaaggccgaagctgatcctcaggctatccccaagaagagaggccggaagcctggatctgtggttgctgctgccgccgcagaggccaagaaaaaggccgtgaaagagtccagcatccgcagcgtgcaagagacagtgctgcccatcaagaagcgcaagacccgggaaaccgtgtccatcgaagtgaaagaggtggtcaagcctctgctggtgtctaccctgggcgagaagtctggcaagggactgaaaacctgcaagtcccctggcagaaagagcaaagagtctagccctaagggcagaagcagcagcgctagctccccacctaagaaagaacaccaccatcaccaccaccatagcgagtccccaaaggctccagttcctctgcttcctccactgcctccacctccacctgagcctgagtctagcgaggatcctacaagccctcctgagccacaggatctgagcagctccgtgtgcaaagaagagaagatgcccagaggcggctccctggaatctgacggctgtcctaaagagcccgccaagacacagcctgccgttgccacagctgcaacagctgccgagaagtacaagcacagaggcgagggcgagcgcaaggatatcgtgtctagcagcatgcccagacctaaccgcgaggaacccgtggatagcagaacccctgtgaccgagagggtgtcctgatgactcgacggcgcgcccatggcgatatccaattggagctcgcggccgcctcgacttaccggtagtgaattctaccagtgccataggatagtgaattctaccagtgccatacacgtgagtgaattctaccagtgccatagcatgcagtgaattctaccagtgccataggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 2026 bp, Artificial Sequence
  mRNA transcript variant
ccggcgtcggcggcgcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagctgcaagatgggattcagatctgttctcaagcctgtcgttccaggacccaggagggagaagcagctgccaggggaagtctcttcgtaggcggaggtcaggagtccaagaggagtgagcagagtcacagaagcctcttaaagcctcttcttcccccatcccatcaacacggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaaaaaaaaaaaaaaaaaaaaaaaaaaaaaaaaaaaaaaaa

SEQ ID NO: 17 - DNA, 10241 bp, Artificial Sequence
  mRNA transcript variant
ccggcgtcggcggcgcgcgcgctccctcctctcggagagagggctgtggtaaaagccgtccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccgtcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaattt

SEQ ID NO: 18 - DNA, 1734 bp, Artificial Sequence
  mRNA transcript variant
ccggcgtcggcggcgcgcgcgctccctcctctcggagagagggctgtggtaaaagccgtccggaaaatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaaaaaaaaaaaaaaaaaaaaaaaaaaaaaaaaaaaaaaaa

SEQ ID NO: 19 - protein, 393 aa, Artificial Sequence
  MECP2 variant
MYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 20 - protein, 486 aa, Artificial Sequence; identical to SEQ ID NO: 8
  MECP2 variant

SEQ ID NO: 21 - protein, 498 aa, Artificial Sequence [sequence omitted]

SEQ ID NO: 22 - DNA, 466 bp, Artificial Sequence
  promoter
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcg

SEQ ID NO: 23 - DNA, 2728 bp, Artificial Sequence
  Expression vector
  repeat_region (1)..(130): 5′ITR
  promoter (213)..(678): hSynapsin
  misc_feature (690)..(695): Kozak
  misc_feature (696)..(2195): human MECP2 alpha
  polyA_signal (2303)..(2534): SV40 late polyadenylation
  repeat_region (2599)..(2728): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagcatgcctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattcgccaccatggctgctgcagctgctgctgcaccttctggtggtggcggaggcggagaggaagagagactggaagagaagtccgaggaccaggatctgcagggcctgaaggacaagcccctgaagttcaagaaagtgaagaaggataagaaagaggaaaaagagggcaagcacgagcccgtgcagccatctgctcaccattctgccgaacctgccgaagccggaaaggccgaaacatctgaaggctctggaagcgcccctgccgtgcctgaagcttctgcttctcctaagcagcggcggagcatcatcagagacagaggccctatgtacgacgaccccacactgcctgaaggctggaccagaaagctgaagcagagaaagagcggcagaagcgccgggaagtacgacgtgtacctgatcaaccctcagggcaaagccttccggtccaaggtggaactgatcgcctacttcgagaaagtgggcgacacctctctggaccccaacgacttcgatttcaccgtgacaggcagaggcagccccagcagaagagagcagaagcctcctaagaagcctaagagccccaaggctcctggcaccggaagaggtagaggtagacctaaaggcagcggcaccacaagacctaaggccgctacttctgagggcgtgcaagtgaagagagtgctggaaaagagccccggcaagctgctggtcaagatgccctttcagacaagccctggcggcaaagcagaaggcggaggggctacaacaagcacccaagtgatggtcatcaagaggcccggcagaaagcggaaggccgaagctgatcctcaggctatccccaagaagagaggccggaagcctggatctgtggttgctgctgccgccgcagaggccaagaaaaaggccgtgaaagagtccagcatccgcagcgtgcaagagacagtgctgcccatcaagaagcgcaagacccgggaaaccgtgtccatcgaagtgaaagaggtggtcaagcctctgctggtgtctaccctgggcgagaagtctggcaagggactgaaaacctgcaagtcccctggcagaaagagcaaagagtctagccctaagggcagaagcagcagcgctagctccccacctaagaaagaacaccaccatcaccaccaccatagcgagtccccaaaggctccagttcctctgcttcctccactgcctccacctccacctgagcctgagtctagcgaggatcctacaagccctcctgagccacaggatctgagcagctccgtgtgcaaagaagagaagatgcccagaggcggctccctggaatctgacggctgtcctaaagagcccgccaagacacagcctgccgttgccacagctgcaacagctgccgagaagtacaagcacagaggcgagggcgagcgcaaggatatcgtgtctagcagcatgcccagacctaaccgcgaggaacccgtggatagcagaacccctgtgaccgagagggtgtcctgatgactcgacggcgcgcccatggcgatatccaattggagctcgcggccgcctcgacttaccggtttgatatcttctagaaggatccaacgcgtaggtaccggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 3948 bp, Artificial Sequence
  Expression Vector
  repeat_region (1)..(130): 5′ITR
  repeat_region (198)..(579): CMV IE promoter
  promoter (582)..(863): CB promoter
  TATA_signal (836)..(839): TATA
  Intron (957)..(1929): chicken beta-actin intron
  misc_feature (1993)..(3486): MECP2 e1
  misc_feature (3490)..(3537): SpA
  polyA_signal (3604)..(3730): Rabbit globin poly A
  repeat_region (3819)..(3948): 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctgtacggaagtgttacttctgctctaaaagctgcggaattgtacccgcgggcccaccatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctagaataaagagctcagatgcatcgatcagagtgtgttggttttttgtgtgacgcgtggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2022/0202960 A1
(43) Pub. Date: Jun. 30, 2022

(54) COMPOSITIONS USEFUL IN TREATMENT OF RETT SYNDROME

(71) Applicant: The Trustees of the University of Pennsylvania, Philadelphia, PA (US)

(72) Inventors: James M. Wilson, Philadelphia, PA (US); Ralf Schmid, Havertown, PA (US)

(21) Appl. No.: 17/605,827

(22) PCT Filed: Apr. 23, 2020
(86) PCT No.: PCT/US2020/029642

§ 371 Date: Oct. 22, 2021

Related U.S. Application Data
(60) Provisional application No. 62/837,947, filed on Apr. 24, 2019.

(51) Int. Cl. A61K 48/00; A61K 38/17; A61P 25/28; C12N 15/86; C12N 15/113

(52) U.S. Cl. CPC A61K 48/0058; A61K 38/177; A61P 25/28; C12N 2310/141; C12N 15/113; C12N 2750/14143; C12N 2750/14171; C12N 15/86

(57) ABSTRACT

Provided is a recombinant adeno-associated virus (rAAV) having an AAV capsid and a vector genome which comprises a nucleic acid sequence encoding a functional human methyl-CpG-binding protein 2 (hMECP2). Also provided is a production system useful for producing the rAAV, a pharmaceutical composition comprising the rAAV, and a method of treating a subject having Rett Syndrome, or ameliorating symptoms of Rett Syndrome, or delaying progression of Rett Syndrome via administrating an effective amount of the rAAV to a subject in need thereof.

### BACKGROUND OF THE INVENTION

Rett syndrome (RTT) is a severe neurodevelopmental disorder (˜1:10,000 live female births) resulting from loss-of-function mutations in the X-linked gene encoding methyl-CpG-binding protein 2 (MECP2) (Amir, R. E. et al. (1999). Rett syndrome is caused by mutations in X-linked MECP2, encoding methyl-CpG-binding protein 2. Nat Genet 23, 185-188, doi:10.1038/13810). After apparently typical early postnatal development, girls affected with RTT exhibit regression of skills around the second year of life, leading to hallmark symptoms such as severe communication deficits (e.g. loss of speech) and motor impairments (e.g. loss of the ability to walk) (Katz, D. M. et al. (2016). Rett Syndrome: Crossing the Threshold to Clinical Translation. Trends in neurosciences 39, 100-113, doi:10.1016/j.tins.2015.12.008). Patients also exhibit life-long respiratory problems, gastrointestinal dysfunction, seizures, anxiety, and orthopedic problems that pose a heavy emotional and financial burden on parents and caretakers.

Current RTT treatments are ineffective, and none have overcome the causative loss of MECP2 function (Katz, D. M. et al., as cited above). In affected females, one of the alleles of the X-chromosome carries a disease-causing MeCP2 mutation, whereas the other allele carries the wildtype allele. Random X-chromosome inactivation (XCI) during development results in a mosaic MECP2 protein expression with cells expressing mutant MeCP2 being diseased. Activation of the wild-type copy of MeCP2 on the inactive allele (Xi) in diseased cells is thought to provide a viable approach to normalize MECP2 protein expression and, consequently, to treat RTT. It had been shown previously that reinstatement of MeCP2 in adult RTT model mice dramatically improves disease symptoms (Guy, J., Gan, J., Selfridge, J., Cobb, S. & Bird, A. (2007). Reversal of neurological defects in a mouse model of Rett syndrome. Science 315, 1143-1147, doi:10.1126/science.1138389). This observation suggests that restoring MeCP2 expression in individuals with RTT may provide a transformative treatment.

Extremely modest levels of MeCP2 activation have been achieved in proliferating, non-neuronal cells by utilizing small-molecule compounds or RNAi technology (Bhatnagar, S. et al. (2014). Genetic and pharmacological reactivation of the mammalian inactive X chromosome. Proc Natl Acad Sci USA 111, 12591-12598, doi:10.1073/pnas.1413620111; and Sripathy, S. et al. (2017). Screening for reactivation of MeCP2 on the inactive X chromosome identifies the BMP/TGF-beta superfamily as a regulator of XIST expression. Proc Natl Acad Sci USA, doi:10.1073/pnas.1621356114). Additional, unpublished small-molecule screens have been performed with non-neuronal and neuronal cells, but no validated leads for MeCP2 activation have emerged.

MECP2 gene therapy has been pursued as an alternative approach to pharmacological therapy attempts. An MECP2 expression cassette with endogenous regulatory elements from the mouse Mecp2 genomic locus packaged into AAV9 and delivered by neonatal intraventricular injection significantly increased the life span and general well-being of a male Rett syndrome model. However, efficacy to correct behavior benchmarks to levels seen in wildtype littermates was limited (Sinnett, S. E. et al. (2017). Improved MECP2 Gene Therapy Extends the Survival of MeCP2-Null Mice without Apparent Toxicity after Intracisternal Delivery. Mol Ther Methods Clin Dev 5, 106-115, doi:10.1016/j.omtm.2017.04.006; and Gadalla, K. K. E. et al. (2017). Development of a Novel AAV Gene Therapy Cassette with Improved Safety Features and Efficacy in a Mouse Model of Rett Syndrome. Mol Ther Methods Clin Dev 5, 180-190, doi:10.1016/j.omtm.2017.04.007). When an artificially truncated and spliced MECP2 transgene was used instead, therapeutic efficacy in mice was improved (Tillotson, R. et al. (2017). Radically truncated MeCP2 rescues Rett syndrome-like neurological defects. Nature, doi:10.1038/nature24058).

Thus, there is an urgent unmet medical need to develop a new approach to achieve sufficient levels of MECP2 protein in neurons for a therapeutic benefit.

### SUMMARY OF THE INVENTION

Provided herein is a therapeutic, recombinant, and replication-defective adeno-associated virus (rAAV) which is useful for treating Rett Syndrome (RTT) in a subject in need thereof. The rAAV carries a vector genome comprising inverted terminal repeats (ITR) and a nucleic acid sequence encoding a functional human methyl-CpG-binding protein 2 (hMECP2) under the control of regulatory sequences which direct the hMECP2 expression in a target cell. In certain embodiment, the rAAV further comprises an AAV capsid in which the vector genome is packaged, for example an AAVhu68 capsid or an AAV-PHP.B capsid. In certain embodiments, the hMECP2-coding sequence is about 95% to 100% identical to SEQ ID NO: 3. Additionally or alternatively, the function hMECP2 protein has an amino acid sequence of SEQ ID NO: 2. In certain embodiments, the hMECP2-coding sequence is SEQ ID NO: 3. In certain embodiments, the vector genome further comprises at least two tandem repeats of dorsal root ganglion (drg)-specific miRNA target sequences. In certain embodiments, the vector genome has a sequence of nucleotide (nt) 1 to nt 2728 of SEQ ID NO: 1 or nt 1 to nt 2802 of SEQ ID NO: 6. In certain embodiments, the rAAV or a composition comprising the rAAV is administrable to a subject in need thereof to ameliorate symptoms of Rett Syndrome and or to delay progression of Rett Syndrome.

In another aspect, a production system useful for producing the rAAV is provided. In this system, cells were cultured which comprises a nucleic acid sequence encoding an AAV capsid protein, a vector genome as described herein and sufficient AAV rep functions and helper functions to permit packaging of the vector genome into the AAV capsid. In certain embodiment, the AAV capsid is a Clade F capsid, e.g., AAVhu68, AAV9, or AAV-PHP.B.

In one aspect, provided herein is a vector which is useful for treating Rett Syndrome (RTT) in a subject in need thereof. The vector carries a nucleic acid sequence encoding a functional human methyl-CpG-binding protein 2 (hMECP2) under the control of regulatory sequences which direct the hMECP2 expression in a target cell. In certain embodiments, the hMECP2-coding sequence is about 95% to 100% identical to SEQ ID NO: 3. Additionally or alternatively, the function hMECP2 protein has an amino acid sequence of SEQ ID NO: 2. In certain embodiments, the hMECP2-coding sequence is SEQ ID NO: 3. In certain embodiments, the vector further carries at least two tandem repeats of dorsal root ganglion (drg)-specific miRNA target sequences. In certain embodiments, the vector or a composition comprising the vector is administrable to a subject in need thereof to ameliorate symptoms of Rett Syndrome and or to delay progression of Rett Syndrome.

In a further aspect, provided herein is a composition comprising a rAAV or a vector as described herein and an aqueous suspension media.

In another aspect, a method of treating a subject having Rett Syndrome, or ameliorating symptoms of Rett Syndrome, or delaying progression of Rett Syndrome is provided. The method comprises administrating an effective amount of a rAAV or a vector as described herein to a subject in need thereof. In certain embodiments, the vector or rAAV is administrable to a patient via an intra-cisterna magna injection (ICM). In certain embodiments, a vector or a composition is provided which is administrable to a patient having Rett Syndrome who is 18 years of age or younger. In certain embodiments, a vector or a composition is provided which is administered to a patient having Rett Syndrome who is 18 years of age or older.

These and other aspects of the invention are apparent from the following detailed description of the invention.

### BRIEF DESCRIPTION OF THE DRAWINGS

FIG. 1 provides a schematic diagram of a plasmid for producing an rAAV comprising an AAV.hSyn.hMECP2co.SV40 vector genome. Full nucleic acid sequence of this plasmid is shown as SEQ ID NO: 1.

FIGS. 2A to 2B provide a schematic diagram of a plasmid for producing an rAAV comprising an AAV.hSyn.hMECP2co.miR183.SV40 vector genome and show successful expression of hMECP2. FIG. 2A provides a schematic diagram of plasmid with full nucleic acid sequence of this plasmid shown as SEQ ID NO: 6 (expression vector SEQ ID NO: 15). FIG. 2B provides a Western Blot showing that MECP2 expression in mouse brain is not affected by the miR183 in expression cassette, when rAAV was administered with an IV injection of 5×10¹¹GC/mouse to a Mecp2-ko mouse cortex at 3 weeks.

FIG. 3 provides a schematic diagram of a plasmid for producing an rAAV comprising an AA V. CB7.CI.hMECP2.rBG vector genome. Full nucleic acid sequence of this plasmid is shown as SEQ ID NO: 4.

FIGS. 4A to 4E show successful expression of hMECP2 in Mecp2-ko mice via the AAV-PHP.B.hSyn-MECP2co vector. FIG. 4A provides a representative image showing cell nuclei in brain cortex of a Mecp2-ko mouse via DAPI staining. FIG. 4B shows that no MECP2 protein was detected in the same microscopic field in FIG. 4A. FIG. 4C is an overlay of the images of FIGS. 4A and 4B. FIG. 4D is a representative image showing MECP2 expression in brain cortex of a wildtype mouse. FIG. 4E is a representative image showing MECP2 expression in brain cortex of a Mecp2-ko mouse treated via the AAV-PHP.B.hSyn-MECP2co vector. See, Example 2 for more details. Scale bar, 100 μm.

FIGS. 5A to 5C provide Kaplan-Meier survival plots (FIG. 5A) and body weights (FIGS. 5B and 5C) of Mecp2-ko mice (HEMI or KO) treated with 3×10¹⁰ GC/mouse, 1×10¹¹ GC/mouse, 2.5×10¹¹ GC/mouse, and 5×10¹¹ GC/mouse of the AAV-PHP.B.hSyn-MECP2co vector. Wildtype littermates and Mecp2-ko mice administrated with only PBS served as controls. See, Example 2 for more details.

FIGS. 6A to 6F show behavior correction and quantification of MECP2 expression in Mecp2-ko mice (HEMI) treated with AAV-PHP.B.hSyn-MECP2co vector. Wildtype littermates and Mecp2-ko mice administrated with only PBS served as controls where applicable. FIG. 6A and FIG. 6B provide ambulatory activity level and rearing activity level tested in the Open Field Assay respectively; while FIG. 6C and FIG. 6D provide time spent in Open Zone and frequency of entering Open Zone tested in the Elevated Zero Maze respectively (rAAV administered at 1×10¹¹ GC/mouse, 2.5×10¹¹ GC/mouse, and 5×10¹¹ GC/mouse). FIG. 6E and FIG. 6F show a representative percentage of neurons semi-automatically quantified from triple-stained immunofluorescent images and plotted as % MECP2+/NeuN+ cell at different treatment doses (FIG. 6E, Cerebral Cortex; FIG. 6F, Hippocampus). See, Example 2 for more details.

FIGS. 7A to 7C show behavior correction of Mecp2-ko mice (HEMI) treated with 1×10¹¹ GC/mouse, and 2.5×10¹¹ GC/mouse of the AAV-PHP.B.hSyn-MECP2co vector. Wildtype littermates and Mecp2-ko mice administrated with only PBS served as controls where applicable. FIG. 7A provides latency to fall tested using rotarod, FIG. 7B shows marbles buried, and FIG. 7C shows spontaneous alteration index tested using the Y-maze. See, Example 2 for more details.

FIGS. 8A to 8B provide representative images of dorsal white matter of juvenile mice post treatment and blots with relative overexpression of MECP2. FIG. 8A provides representative images of Luxol Fast staining of dorsal white matter of juvenile wt mice treated with an increasing dose of AAV.hSyn.hMECP2 (Group 1, 3×10⁹ GC/mouse; Group 2, 1×10¹⁰ GC/mouse; Group 3, 5×10¹⁰ GC/mouse; Group 4, 1×10¹¹ GC/mouse; Group 5, 5×10¹¹ GC/mouse; Group 6, 1×10¹² GC/mouse; and Group 7, 5×10¹² GC/mouse). FIG. 8B provides with a graph of relative overexpression of MECP2 in WT brains after AAV vector injection at different doses (as quantified from Western blots). See, Example 2 for more details.

FIG. 9 shows cells and MECP2 positive cells in pyramidal layer of hippocampus, grey matter of spinal cord, and dorsal root ganglion cells (DRG) of wild type mice with or without the vector treatment.

FIGS. 10A to 10C provide ambulatory activity (FIG. 10A), rearing activity (FIG. 10B) tested in Open Filed Assay and latency to fall tested using a rotarod (FIG. 10C) on adult wt mice treated with 1×10¹² GC/mouse AAV.hSyn.hMECP2 or only PBS. See, Example 2 for more details.

FIG. 11 provides representative images showing axonopathy in dorsal white matter tracts observed in rhesus macaques treated with AAVhu68.MECP vectors. See, Example 3 for more details.

FIG. 12 shows mild to moderate loss of myelin in Spinal White Matter Tracts in the tested groups. See, Example 3 for more details.

FIGS. 13A to 13D provide vector biodistribution across various tissues. FIG. 13A shows biodistribution of the AAVhu68.MeP426.MECP2.RDH1.Stuffer vector. FIG. 13B shows biodistribution of the AAVhu68.MeP426.MECP2-myc.RDH1.Stuffer vector. FIG. 13C shows biodistribution of the AAVhu68sc.mMeP546.SVI.MeCP2e1.SpA vector. FIG. 13D shows biodistribution of the AAVhu68.CB7.CI.MECP2.rBG vector. See Example 3 for more details.

FIG. 14 shows successful expression of hMECP2 in both brain cortex and dorsal root ganglia of spinal cord in Group 2 using anti-myc antibodies. See, Example 3 for more details.

FIG. 15 shows successful expression of hMECP2 in both brain cortex and dorsal root ganglia of spinal cord in Groups 2 and 4 using anti-MECP2 antibodies. See, Example 3 for more details.

FIGS. 16A to 16C provide biodistribution across various tissues of the AAVhu68.hSyn.MECP2co.SV40 vector administrated at different doses (FIG. 16A, 3×10¹³ GC/NHP; FIG. 16B, 1×10¹³ GC/NHP; and FIG. 16C, 3×10¹² GC/NHP). See, Example 3 for more details.

FIG. 17 provides relative expression of hMECP2 across various tissues achieved by the AAVhu68.hSyn.MECP2co.SV40 vector. See, Example 3 for more details.

FIGS. 18A to 18C show reduction in axonopathy in non-human primates (NHP) with DRG de-targeting via miR183. FIG. 18A provides with representative images of MECP2 expression in DRG cells (in-situ hybridization, MECP2 shown in red, nuclei shown in blue). FIG. 18B (spinal cord) and FIG. 18C (DRG) provide graphs with pathological scoring of white matter tracks and DRG from NHP injected with a high dose of AAVhu68.hSYN.MECP2co either with or without miR183 (N=3/group, tissue harvested after 3 months). See, Example 3 for more details.

### DETAILED DESCRIPTION OF THE INVENTION

Compositions and methods for treating Rett Syndrome are provided herein. An effective amount of a recombinant adeno-associated virus (rAAV) having an AAV capsid (e.g., AAVhu68 or AAV-PHP.B) and packaged therein a vector genome encoding a functional human methyl-CpG binding protein 2 (hMECP2) is delivered to a subject in need.

I. Human Methyl-CpG Binding Protein 2 (hMECP2)

Methyl-CpG Binding Protein 2 (MECP2, MeCP2 or MeCp2) is a chromosomal protein that binds to methylated DNA and then interacts with other proteins (for example, histone deacetylase, the corepressor SIN3A, or the transcription factor CREB1) to form a complex that turns off a gene or to act as a transcriptional activator. Two isoforms of Human MECP2 (hMECP2) protein (UniProtKB-P51608, MECP2_HUMAN) have been identified: hMECP2 Isoform A also known as hMECP2Beta or hMECP2-e2(P51608-1, SEQ ID NO: 8) and Isoform B also known as hMECP2alpha or hMECP2-e1 (P51608-2, SEQ ID NO: 2). In certain embodiments, MECP2 and hMECP2 may be used interchangeably when referring to a human MECP2 protein.

As used herein, a functional hMECP2 protein refers to an isoform, a natural variant, a variant, a polymorph, or a truncation of a MECP2 protein which is not associated with Rett Syndrome and/or delivery or expression of which may ameliorate symptoms or delay progression of Rett Syndrome in an animal model or a patient. See, OMIM #312750 (omim org/entry/312750), genecards.org/cgi-bin/carddisp.p1?gene=MECP2 and uniprot.org/uniprot/P51608, each of the webpages is incorporated herein by reference in its entirety. In certain embodiments, the functional hMECP2 protein has an amino acid sequence of SEQ ID NO: 2 or an amino acid sequence at least about 90% (e.g., at least about 90%, 91%, 92%, 93%, 94%, 95%, 96%, 97%, 98%, 99%, or 99.9%) identical thereto. In certain embodiments, the functional hMECP2 protein has an amino acid sequence of SEQ ID NO: 2 or an amino acid sequence at least about 78% to at least about 80% identical thereto. In certain embodiment, the functional hMECP2 protein has an amino acid sequence of SEQ ID NO: 8 or an amino acid sequence at least about 90% (e.g., at least about 90%, 91%, 92%, 93%, 94%, 95%, 96%, 97%, 98%, 99%, or 99.9%) identical thereto. In certain embodiments, the functional hMECP2 protein has an amino acid sequence of NCBI Reference Sequences NP_001303266.1 (SEQ ID NO: 19), NP_004983.1 (SEQ ID NO:20), or NP_001104262.1 (SEQ ID NO: 21). In certain embodiment, the functional hMECP2 is a truncated hMECP2 which comprises a methyl-CpG binding domain (MBD) having the sequence and a NCoR/SMRT Interaction Domain (NID). See, WO2018172795A1, which is incorporated herein by reference in its entirety.

In certain embodiments, a functional hMECP2 protein ameliorates symptoms or delays progression of Rett Syndrome in an RTT animal model. One exemplified RTT animal model is a Mecp2-ko mouse. In one embodiment, the RTT animal model is a male hemizygous Mecp2-ko mouse. The RTT symptoms or progression may be evaluated using various assays/methods, including but not limited to, a survival plot (e.g., Kaplan-Meier survival plot), monitoring body weights, and observing behavior changes (for example, by Open Field Assay, Elevated Zone Maze, Y Maze, Marble Burying Assay, and rotarod assay). In certain embodiment, administration or expression of a functional hMECP2 protein in an RTT animal model leads to amelioration of RTT symptoms or delay in RTT progression shown by an assay result which is at least about 10%, 20%, 30%, 40%, 50%, 60%, 70%, 80%, 90%, 95%, 100%, or more than 100% of that obtained in a corresponding wildtype animal. In certain embodiment, administration or expression of a functional hMECP2 protein in an RTT animal model leads to amelioration of RTT symptoms or delay in RTT progression shown by an improved assay result which is at least about 10%, 20%, 30%, 40%, 50%, 60%, 70%, 80%, 90%, 95%, 100%, or more than 100% of that obtained from a corresponding non-treated RTT animal. An illustration is detailed in Example 2.

Provided herein is a nucleic acid sequence encoding a functional hMECP2 protein, termed as hMECP2 coding sequence or MECP2 coding sequence. In certain embodiments, the hMECP2 coding sequence is selected from SEQ ID NO: 3 (referred to as MECP2 or MECP2co) or NCBI Reference Sequences NM_001110792.1 (referred to as MECP2 or MECP2e1; SEQ ID NO: 18) encoding for amino acid sequence NP_001104262.1 (SEQ ID NO: 21), NM_001316337.1 (SEQ ID NO: 16) encoding for amino acid sequence NP_001303266.1 (SEQ ID No: 19), NM 004992.3 (SEQ ID NO: 17) encoding for amino acid sequence NP_004983.1 (SEQ ID NO: 20), GQ203295.1, HQ141378.1, GQ203293.1, HM156733.1, GQ203294.1, GQ896382.1, GU479943.1, HM156732.1, HM020402.1, AF158180.1, AJ132917.1, AB209464.1, X89430.1, AK289444.1, BX538060.1, BC011612.1, L37298.1, Y12643.1, X99686.1, AY541280.1, GU812285.1, GU812286.1, HQ141377.1, HQ127345.1, DQ656049.2, HQ154629.1, DQ656051.2, BC031833.1, BI767019.1, HM005664.1, KU178174.1, KU178175.1, KU178176.1, KU178177.1, KU178178.1, KU178179.1, KU178180.1, or a nucleic acid sequence at least about 70% (e.g., at least about 75%, 80%, 85%, 90%, 91%, 92%, 93%, 94%, 95%, 96%, 97%, 98%, 99%, or 99.9%) identical thereto. Each of the NCBI Reference Sequences is incorporated herein by reference in its entirety. In certain embodiments, the hMECP2 coding sequence is a modified or engineered (hMECP2 or hMECP2co). The modified or engineered (hMECP2 or hMECP2co) shares less than about 70% (e.g., about 75%, 80%, 85%, 90%, 91%, 92%, 93%, 94%, 95%, 96%, 97%, 98%, 99%, or 99.9%) identity to the NCBI Reference Sequences. In certain embodiments, the hMECP2 coding sequence is SEQ ID NO: 3 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto. In certain embodiments, the coding sequence having the recited identity to SEQ ID NO: 3 encodes an amino acid of SEQ ID NO: 2. In certain embodiments, the coding sequence having the recited identity to SEQ ID NO: 3 does not encode the protein of SEQ ID NO: 16. In certain embodiments, the nucleic acid sequence having the recited identity to SEQ ID NO: 3 does not encode the protein to SEQ ID NO: 17. In certain embodiments, the nucleic acid sequence having the recited identity to SEQ ID NO: 3 does not encode SEQ ID NO: 18.

In certain embodiments, the hMECP2 coding sequence is SEQ ID NO: 18 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto, which encodes the amino acid sequence of SEQ ID NO: 21.

In certain embodiments, the hMECP2 coding sequence is SEQ ID NO: 16 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto, which encodes the amino acid sequence of SEQ ID NO: 19.

In certain embodiments, the hMECP2 coding sequence is SEQ ID NO: 17 or a nucleic acid sequence at least about 70% (e.g., at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.9%) identical thereto, which encodes the amino acid sequence of SEQ ID NO: 20.

A “nucleic acid”, as described herein, can be RNA, DNA, or a modification thereof, and can be single or double stranded, and can be selected, for example, from a group including: nucleic acid encoding a protein of interest, oligonucleotides, nucleic acid analogues, for example peptide-nucleic acid (PNA), pseudocomplementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

II. Rett Syndrome

MECP2 gene mutations are the cause of most cases of Rett syndrome, a progressive neurologic developmental disorder and one of the most common causes of cognitive disability in females. Males who have the genetic mutation that causes Rett syndrome are affected in devastating ways. Most of them die before birth or in early infancy. See, e.g., ninds.nih.gov/Disorders/Patient-Caregiver-Education/Fact-Sheets/Rett-Syndrome-Fact-Sheet and omim.org/entry/312750.

“Patient” or “subject”, as used herein interchangeably, means a male or female mammalian animal, including a human, a veterinary or farm animal, a domestic animal or pet, and animals normally used for clinical research. In one embodiment, the subject of these methods and compositions is a human patient. In one embodiment, the subject of these methods and compositions is a male or female human. In certain embodiment, the subject of these methods and compositions is diagnosed with Rett Syndrome and/or with symptoms of Rett Syndrome.

The methods and compositions may be used for treatment of any of the following four stages of Rett syndrome: Stage I, called early onset, typically begins between 6 and 18 months of age; Stage II, or the rapid destructive stage, usually begins between ages 1 and 4 and may last for weeks or months; Stage III, or the plateau or pseudo-stationary stage, usually begins between ages 2 and 10 and can last for years; and Stage IV, or the late motor deterioration stage, can last for years or decades. In certain embodiments, the subject is a human less than 18 years old (e.g., less than about 1, 2, 3, 4, 5, 6, 7, 8, 9, 10, 11 month(s) old, or less than about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old). Additionally or alternatively, the subject is a newborn or a human more than 1 month old (e.g., more than about 2, 3, 4, 5, 6, 7, 8, 9, 10, 11 month(s) old, or more than about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old). In certain embodiments, the patient is about 1, 2, 3, 4, 5, 6, 7, 8, 9, 10, 11 month(s) old, or about 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18 year(s) old. In certain embodiments, the patient is a toddler, e.g., 18 months to 3 years of age. In certain embodiments, the patient is from 3 years to 6 years of age, from 3 years to 12 years of age, from 3 years to 18 years of age, from 3 years to 30 years of age. In certain embodiments, patients are 18 years of age or older, or are older than 18 years of age.

Symptoms of Rett Syndrome may include, but are not limited to the following: normal early growth and development followed by a slowing of development, loss of muscle tone (hypotonia), difficulty feeding, and jerkiness in limb movements, loss of purposeful use of the hands, distinctive hand movements, problems crawling or walking, diminished eye contact, autistic-like behaviors, walking on the toes, sleep problems, a wide-based gait, teeth grinding and difficulty chewing, slowed growth, seizures, cognitive disabilities, and breathing difficulties while awake such as hyperventilation, apnea (breath holding), and air swallowing, Apraxia (the inability to perform motor functions, including eye gaze and speech), delays in gross motor skills such as sitting or crawling, slowed brain and head growth, compulsive hand movements such as wringing and washing, problems with walking, seizures, and intellectual disability.

As described above, the terms “increase” “decrease” “reduce” “ameliorate” “improve” “delay” or any grammatical variation thereof, or any similar terms indication a change, means a variation of about 5 fold, about 2 fold, about 1 fold, about 90%, about 80%, about 70%, about 60%, about 50%, about 40%, about 30%, about 20%, about 10%, about 5% compared to the corresponding reference (e.g., untreated control or a subject in normal condition without RTT), unless otherwise specified.

In certain embodiments, the patient receives medications controlling some signs and symptoms associated with the RTT, such as seizures, muscle stiffness, or problems with breathing, sleep, the gastrointestinal tract or the heart.

Optionally, an immunosuppressive co-therapy may be used in a subject in need Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 3, 4, 5, 6, 7, or more days prior to or after the gene therapy administration. Such immunosuppressive therapy may involve administration of one, two or more drugs (e.g., glucocorticoids, prednelisone, micophenolate mofetil (MMF) and/or sirolimus (i.e., rapamycin)). Such immunosuppressive drugs may be administrated to a subject in need once, twice or for more times at the same dose or an adjusted dose. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

III. Expression Cassette

Provided herein is a nucleic acid sequence comprising the hMECP2 coding sequence under control of regulatory sequences which direct the hMECP2 expression in a target cell, also termed as an expression cassette. As used herein, an “expression cassette” refers to a nucleic acid molecule which comprises a coding sequence (e.g., a hMECP2 coding sequence), promoter, and may include other regulatory sequences therefor. The regulatory sequences necessary are operably linked to the hMECP2 coding sequence in a manner which permits its transcription, translation and/or expression in target cell. As used herein, “operably linked” sequences include both expression control sequences that are contiguous with the hMECP2 coding sequence and expression control sequences that act in trans or at a distance to control the hMECP2 coding sequence. Such regulatory sequences typically include, e.g., one or more of a promoter, an enhancer, an intron, a Kozak sequence, a polyadenylation sequence, and a TATA signal. In certain embodiment, the promoter is a tissue-specific promoter, e.g., a CNS-specific or neuron-specific promoter. In certain embodiments, the promoter is a human synapsin promoter (also termed hSyn or Syn herein). In certain embodiments, an additional or alternative neuron-specific promoter sequence may be selected from neuron-specific enolase (NSE) promoter (Andersen et al., (1993) Cell. Mol. Neurobiol., 13:503 15), neurofilament light chain gene promoter (Piccioli et al., (1991) Proc. Natl. Acad. Sci. USA, 88:56115), neuron-specific vgf gene promoter (Piccioli et al., (1995) Neuron, 15:373 84), and/or others. In certain embodiments, the human Synaspin promoter has the sequence of (e.g., nt 213 to nt 678 of SEQ ID NO: 1, or SEQ ID NO: 22). Additionally, or alternatively, a chicken beta actin promoter with a cytomegalovirus enhancer (CB7) promoter may be selected. Such a CB7 promoter may have a sequence of, e.g., nt 198 to nt 863 of SEQ ID NO:4, or SEQ ID NO: 12. In certain embodiments, a human elongation initiation factor 1 alpha promoter (EF1a) promoter may be selected. Such a EF1a promoter may have a sequence of, e.g., SEQ ID NO: 13. In certain embodiments, a human ubiquitin C (UbC) promoter or a MeP426 promoter, or a MEP546 promoter for expression of hMECP2. However, in certain embodiments, other promoters, or an additional promoter, may be selected. In certain embodiments, the regulatory sequences direct hMECP2 expression in central nervous system cells.

In certain embodiment, the target cell may be a central nervous system cell. In certain embodiments, the target cell is one or more of an excitatory neuron, an inhibitory neuron, a glial cell, a cortex cell, a frontal cortex cell, a cerebral cortex cell, a spinal cord cell. In certain embodiments, the target cell is a peripheral nervous system (PNS) cell, for example a retina cell. Other cells other than those from nervous system may also be chosen as a target cell, such as a monocyte, a B lymphocyte, a T lymphocyte, a NK cell, a lymph node cell, a tonsil cell, a bone marrow mesenchymal cell, a stem cell, a bone marrow stem cell, a heart cell, an epithelium cell, a esophagus cell, a stomach cell, a fetal cut cell, a colon cell, a rectum cell, a liver cell, a kindly cell, a lung cell, a salivary gland cell, a thyroid cell, an adrenal cell, a breast cell, a pancreas cell, an islet of Langerhans cell, a gallbladder cell, a prostate cell, a urinary bladder cell, a skin cell, a uterus cell, a cervix cell, a testis cell, or any other cell which expresses a functional MECP2 protein in a subject without RTT. See, genecards.org/cgi-bin/carddisp.p1?gene=MECP2&keywords=mecp2#expression.

In certain embodiments, an additional or alternative promoter sequence may be included as part of the expression control sequences (regulatory sequences), e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

In addition to a promoter, a vector may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. An example of a suitable enhancer is the CMV enhancer. Other suitable enhancers include those that are appropriate for desired target tissue indications. In one embodiment, the regulatory sequences comprise one or more expression enhancers. In one embodiment, the regulatory sequences contain two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. In certain embodiments, the intron is a chimeric intron (CI)—a hybrid intron consisting of a human beta-globin splice donor and immunoglobulin G (IgG) splice acceptor elements. Other suitable introns include those known in the art, e.g., such as are described in WO 2011/126808. Examples of suitable polyA sequences include, e.g., Rabbit globin poly A, SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619). In certain embodiments, no WPRE sequence is present.

IV. miRNA

In certain embodiments, in addition to the hMECP2 coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

As used herein, an “miRNA target sequence” is a sequence located on the DNA positive strand (5′ to 3′) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. The term “miR183 cluster target sequence” refers to a target sequence that responds to one or members of the miR183 cluster (alternatively termed family), including miRs-183, -96 and -182 (as described by Dambal, S. et al. Nucleic Acids Res 43:7173-7188, 2015, which is incorporated herein by reference). Without wishing to be bound by theory, the messenger RNA (mRNA) for the transgene (encoding the gene product) is present in a cell type to which the expression cassette containing the miRNA is delivered, such that specific binding of the miRNA to the 3′ UTR miRNA target sequences results in mRNA silencing and cleavage, thereby reducing or eliminating transgene expression only in the cells that express the miRNA.

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides, and contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA.

In certain embodiments, the miRNA target sequence for the at least first and/or at least second miRNA target sequence for the expression cassette mRNA or DNA positive strand is selected from (i) AGTGAATTCTACCAGTGCCATA (miR183, SEQ ID NO: 7); (ii) AGCAAAAATGTGCTAGTGCCAAA (miR-96, SEQ ID NO: 9), (iii) AGTGTGAGTTCTACCATTGCCAAA (miR182, SEQ ID NO: 10). In other embodiments, AGGGATTCCTGGGAAAACTGGAC (SEQ ID NO: 11) is selected.

In certain embodiments, the vector genome or expression cassette contains at least one miRNA target sequence that is a miR-183 target sequence. In certain embodiments, the vector genome or expression cassette contains an miR-183 target sequence that includes AGTGAATTCTACCAGTGCCATA (SEQ ID NO:7), where the sequence complementary to the miR-183 seed sequence is underlined. In certain embodiments, the vector genome or expression cassette contains more than one copy (e.g. two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 7 and, thus, when aligned to SEQ ID NO: 7, there are one or more mismatches. In certain embodiments, a miR-183 target sequence comprises a sequence having at least 1, 2, 3, 4, 5, 6, 7, 8, 9, or 10 mismatches when aligned to SEQ ID NO: 7, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 1, i.e., a sequence that lacks at least 1, 2, 3, 4, 5, 6, 7, 8, 9, or 10 nucleotides at either or both the 5′ or 3′ ends of SEQ ID NO: 1. In certain embodiments, the expression cassette or vector genome comprises a transgene and one miR-183 target sequence. In yet other embodiments, the expression cassette or vector genome comprises at least two, three or four miR-183 target sequences.

In certain embodiments, the vector genome or expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 10). In certain embodiments, the vector genome or expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 10 and, thus, when aligned to SEQ ID NO: 10, there are one or more mismatches. In certain embodiments, a miR-183 target sequence comprises a sequence having at least 1, 2, 3, 4, 5, 6, 7, 8, 9, or 10 mismatches when aligned to SEQ ID NO: 10, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 10, i.e., a sequence that lacks at least 1, 2, 3, 4, 5, 6, 7, 8, 9, or 10 nucleotides at either or both the 5′ or 3′ ends of SEQ ID NO: 10. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

See, PCT/US19/67872, filed Dec. 20, 2019, which is incorporated by reference herein and which claims priority to US Provisional U.S. Patent Application No. 62/783,956, filed Dec. 21, 2018, which is hereby incorporated by reference.

In certain embodiments, the vector genome further comprises at least one, at least two, at least three or preferably at least four tandem repeats of dorsal root ganglion (drg)-specific miRNA target sequences. The target miRNA sequences may be selected from SEQ ID NO: 7 SEQ ID NO: 9, SEQ ID NO: 10, and/or SEQ ID NO: 11. In certain embodiments, the vector genome has a sequence of nucleotide (nt) 1 to nt 2728 of SEQ ID NO: 1 (or SEQ ID NO: 23), nt 1 to nt 2802 of SEQ ID NO: 6 (or SEQ ID NO: 15), and/or nt 1 to nt 3949 of SEQ ID NO: 4 (or SEQ ID NO: 24) or a nucleic acid sequence at least about 70% (e.g., at least about 75%, 80%, 85%, 90%, 91%, 92%, 93%, 94%, 95%, 96%, 97%, 98%, 99%, or 99.9%) identical thereto, which further comprises at least one, at least two, at least three and or at least four miRNA drg de-targeting sequence. In certain embodiments, the vector genome comprises the sequence of SEQ ID NO: 3 or a sequence at least 95%, at least 96%, at least 97%, at least 98%, at least 99 to at least 100% identical thereto, which further comprises at least one miRNA drg de-targeting sequence, at least two, at least three and or at least 4. The target miRNA sequences may be selected from SEQ ID NO: 7, SEQ ID NO: 9, SEQ ID NO: 10, and/or SEQ ID NO: 11.

V. rAAV

Provided herein is a recombinant adeno-associated virus (rAAV) useful for treating Rett Syndrome. The rAAV comprises (a) an AAV capsid; and (b) a vector genome packaged in the AAV capsid of (a). Suitably, the AAV capsid selected targets the cells to be treated. In certain embodiments, the capsid is from Clade F. However, in certain embodiments, another AAV capsid source may be selected. The vector genome comprises inverted terminal repeats (ITR) and a nucleic acid sequence encoding a functional human methyl-CpG binding protein 2 (hMECP2) under control of regulatory sequences which direct the hMECP2 expression. In certain embodiments, the hMECP2-coding sequence is at least about 95% identical to SEQ ID NO: 3. In certain embodiments, the hMECP-coding sequence is less than 80% identical to any one of hMECP transcript variants 1 to 3 (NM_001316337.1 (SEQ ID NO: 16), NM_004992.3 (SEQ ID NO: 17) and NM 001110792.1 (SEQ ID NO: 18)). In certain embodiments, the functional hMECP2 has an amino acid sequence of SEQ ID NO: 2. In certain embodiments, the regulatory sequences direct hMECP2 expression in central nervous system cells. In certain embodiments, the regulatory sequences comprise a CNS-specific promoter, e.g., human Synaspin promoter (hSyn), or a constitutive promoter, e.g., CB7. In certain embodiments, the regulatory elements comprise one or more of a Kozak sequence, a polyadenylation sequence, an intron, an enhancer, and a TATA signal. In certain embodiments, the vector genome is nt 1 to nt 2728 of SEQ ID NO: 1, or nt 1 to nt 3949 of SEQ ID NO: 4, or nt 1 to nt 2802 of SEQ ID NO: 6 or a nucleic acid sequence at least about 70% (e.g., at least about 75%, 80%, 85%, 90%, 91%, 92%, 93%, 94%, 95%, 96%, 97%, 98%, 99%, or 99.9%) identical thereto. In certain embodiments, the vector genome further comprises dorsal root ganglion (drg)-detargetting miRNA target sequences as described herein.

In certain embodiments, the Clade F AAV capsid is selected from an AAVhu68 capsid, an AAV9 capsid, an AAVhu32 capsid, or an AAVhu31 capsid. Nucleic acid sequences encoding a suitable capsid may be utilized in the production of the AAV.hMECP2 recombinant AAV (rAAV) carrying the vector genome. Additional details relating to AAVhu68 are provided in WO 2018/160582, and US 2015/0079038, each of which is incorporated herein by reference in its entirety. The Clade F vectors described herein are well suited for delivery of the vector genome comprising the hMECP2 coding sequence to cells within the central nervous system, including brain, hippocampus, motor cortex, cerebellum, and motor neurons. These vectors may be used for targeting other cells within the central nervous system (CNS) and certain other tissues and cells outside the CNS. In other embodiments, a Clade A capsid (e.g., AAV1) may be selected. Still other AAV or other parvovirus capsids may be selected.

In certain embodiments, the AAV capsid for the compositions and methods described herein is chosen based on the target cell. In certain embodiment, the AAV capsid transduces a CNS cell and/or a PNS cell. In certain embodiments, the AAV capsid is selected from a cy02 capsid, a rh43 capsid, an AAV8 capsid, a rh01 capsid, an AAV9 capsid, an rh8 capsid, a rh10 capsid, a bb01 capsid, a hu37 capsid, a rh02 capsid, a rh20 capsid, a rh39 capsid, a rh64 capsid, an AAV6 capsid, an AAV1 capsid, a hu44 capsid, a hu48 capsid, a cy05 capsid a hu11 capsid, a hu32 capsid, a pi2 capsid, or a variation thereof. In certain embodiments, the AAV capsid is a Clade F capsid, such as AAV9 capsid, AAVhu68 capsid, AAV-PHP.B capsid, hu31 capsid, hu32 capsid, or a variation thereof. See, e.g., WO 2005/033321 published Apr. 14, 2015, WO 2018/160582, and US 2015/0079038, each of which is incorporated herein by reference in its entirety. In certain embodiments, the AAV capsid is a non-clade F capsid, for example a Clade A, B, C, D, or E capsid. In certain embodiment, the non-Clade F capsid is an AAV1 or a variation thereof. In certain embodiment, the AAV capsid transduces a target cell other than the nervous system cells. In certain embodiments, the AAV capsid is a Clade A capsid (e.g., AAV1, AAV6), a Clade B capsid (e.g., AAV 2), a Clade C capsid (e.g., hu53), a Clade D capsid (e.g., AAV7), or a Clade E capsid (e.g., rh10). Still, other AAV capsid may be chosen.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321.

As indicated above, a rAAV is provided which has an AAV capsid which targets the desired cells and a vector genome which comprises, at a minimum, AAV ITRs required to package the vector genome into the capsid, a hMECP2 coding sequence and regulatory sequences which direct expression therefor. In certain embodiments, the vector genome is a single-stranded AAV vector genome. In certain embodiments, a rAAV vector may be utilized in the invention which contains self-complementary (sc) AAV vector genome.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed ΔITR, has been described in which the D-sequence and terminal resolution site (trs) are deleted. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In certain embodiments, vector genomes are constructed which comprise a 5′ AAV ITR-promoter-optional enhancer-optional intron-hMECP2 coding sequence-polyA-3′ ITR. In certain embodiments, an enhancer may be located downstream of the hMECP2 coding sequence. In certain embodiments, the vector genome further comprises an drg detargetting sequence, such as miR183 or miR182 as described herein. In certain embodiments, two, three, four or more copies of such drg detargetting sequences may be present in the vector genome (e.g., in the 3′ untranslated region (UTR) between the hMECP2 stop codon(s) and the polyA) as describe in the specification. In certain embodiments, the rAAV is a single-stranded AAV. In certain embodiments, the rAAV is a self-complementary AAV. In certain embodiments, the ITRs are from AAV2. In certain embodiments, more than one promoter is present. In certain embodiments, the promoter is Synapsin (hSyn). In certain embodiments, the promoter is CB7. In certain embodiments, the promoter is neuron-specific enolase (NSE) promoter. In certain embodiments, the promoter is neurofilament light chain gene promoter. In certain embodiments, the promoter is neuron-specific vgf gene promoter. In certain embodiments, the enhancer is present in the vector genome. In certain embodiments, more than one enhancer is present. In certain embodiments, an intron is present in the vector genome. In certain embodiments, the enhancer and intron are present. In certain embodiments, the intron is a chimeric intron (CI)—a hybrid intron consisting of a human beta-globin splice donor and immunoglobulin G (IgG) splice acceptor elements. In certain embodiments, the polyA is an SV40 poly A (i.e., a polyadenylation (PolyA) signal derived from Simian Virus 40 (SV40) late genes). In certain embodiments, the polyA is a rabbit beta-globin (RBG) poly A. In certain embodiments, the vector genome comprises a 5′ AAV ITR-hSyn promoter-hMECP2 coding sequence-poly A-3′ ITR. In certain embodiments, the vector genome comprises a 5′ AAV ITR-CB7 promoter-hMECP2 coding sequence-RBG poly A-3′ ITR. In certain embodiments, an enhancer may be located downstream of the hMECP2 coding sequence. In certain embodiments, the vector genome further comprises an drg detargetting sequence, such as miR183 or miR182 as described herein. In certain embodiments, two, three, four or more copies of such drg detargetting sequences may be present in the vector genome (e.g., in the 3′ untranslated region (UTR) between the hMECP2 stop codon(s) and the polyA) as describe in the specification. In certain embodiments, the rAAV is a single-stranded AAV. In certain embodiments, the rAAV is a self-complementary AAV.

As used herein, a vector genome or a rAAV comprising the vector genome is illustrated herein as AAV 5′ ITR-promoter (optional)-Kozak (optional)-intron (optional)-MECP2 coding sequence (e.g., hMECP2, hMECP2co, MECP2, MECP2co)-miRNA (optional 0-4+)-polyA(optional)-Stuffer (optional)-AAV 3′ ITR. In certain embodiments, a rAAV is illustrated herein as AAV 5′ ITR-promoter (optional)-Kozak (optional)-intron (optional)-MECP2 coding sequence-miRNA (optionally, 0-4+)-polyA (optional)-Stuffer (optional)-AAV 3′ ITR.

Additionally, provided herein, is an rAAV production system useful for producing a rAAV as described herein. The production system comprises a cell culture comprising (a) a nucleic acid sequence encoding an AAV capsid protein; (b) the vector genome; and (c) sufficient AAV rep functions and helper functions to permit packaging of the vector genome into the AAV capsid. In certain embodiments, the vector genome is nt 1 to nt 2728 of SEQ ID NO: 1, or nt 1 to nt 3949 of SEQ ID NO: 4, or nt 1 to nt 2802 of SEQ ID NO: 6. In certain embodiments, the cell culture is a human embryonic kidney 293 cell culture. In certain embodiments, the AAV rep is from a different AAV. In certain embodiments, wherein the AAV rep is from AAV2. In certain embodiments, the AAV rep coding sequence and cap genes are on the same nucleic acid molecule, wherein there is optionally a spacer between the rep sequence and cap gene. In certain embodiments, the spacer is atgacttaaaccaggt (SEQ ID NO: 14).

For use in producing an AAV viral vector (e.g., a recombinant (r) AAV), the vector genomes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99: 119-145; Buning et al., 2008, Recent developments in adeno-associated virus vector technology, J. Gene Med. 10:717-733; and the references cited below, each of which is incorporated herein by reference in its entirety. For packaging a gene into virions, the ITRs are the only AAV components required in cis in the same construct as the nucleic acid molecule containing the gene. The cap and rep genes can be supplied in trans.

In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, N.Y. (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV is provided. Such a cell culture contains a nucleic acid which expresses the AAV capsid protein in the host cell, a nucleic acid molecule suitable for packaging into the AAV capsid, e.g., a vector genome which contains AAV ITRs and a non-AAV nucleic acid sequence encoding a gene operably linked to regulatory sequences which direct expression of the gene in a host cell; and sufficient AAV rep functions and adenovirus helper functions to permit packaging of the vector genome into the recombinant AAV capsid. In one embodiment, the cell culture is composed of mammalian cells (e.g., human embryonic kidney 293 cells, among others) or insect cells (e.g., Spodoptera frugiperda (Sf9) cells). In certain embodiments, baculovirus provides the helper functions necessary for packaging the vector genome into the recombinant AAV capsid.

Optionally the rep functions are provided by an AAV which cross-complements the capsid. In certain embodiments, at least parts of the rep functions are from AAVhu68B. In another embodiment, the rep protein is a heterologous rep protein other than AAVhu68rep, for example but not limited to, AAV1 rep protein, AAV2 rep protein, AAV3 rep protein, AAV4 rep protein, AAV5 rep protein, AAV6 rep protein, AAV7 rep protein, AAV8 rep protein; or rep 78, rep 68, rep 52, rep 40, rep68/78 and rep40/52; or a fragment thereof; or another source. Any of these AAVhu68 or mutant AAV capsid sequences may be under the control of exogenous regulatory control sequences which direct expression thereof in a host cell.

In one embodiment, cells are manufactured in a suitable cell culture (e.g., HEK 293 or Sf9) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV vector genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in WO 2017/160360, International Patent Application No. PCT/US2016/065970, filed Dec. 9, 2016 and its priority documents, US Patent Application Nos. 62/322,071, filed Apr. 13, 2016 and 62/226,357, filed Dec. 11, 2015 and entitled “Scalable Purification Method for AAV9”, which is incorporated by reference herein.

To calculate empty and full particle content, VP3 band volumes for a selected sample (e.g., in examples herein an iodixanol gradient-purified preparation where # of genome copies (GC)=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL−GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, colorimetric changes, or most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. Endpoint assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAVhu68 particles having packaged genomic sequences from genome-deficient AAVhu68 intermediates involves subjecting a suspension comprising recombinant AAVhu68 viral particles and AAVhu68 capsid intermediates to fast performance liquid chromatography, wherein the AAVhu68 viral particles and AAVhu68 intermediates are bound to a strong anion exchange resin equilibrated at a pH of about 10.2, and subjected to a salt gradient while monitoring eluate for ultraviolet absorbance at about 260 nanometers (nm) and about 280 nm. Although less optimal for rAAVhu68, the pH may be in the range of about 10.0 to 10.4. In this method, the AAVhu68 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/hu68 serotype. Under these ionic conditions, a significant percentage of residual cellular DNA and proteins flow through the column, while AAV particles are efficiently captured.

The rAAV.hMECP2 is suspended in a suitable physiologically compatible composition (e.g., a buffered saline). This composition may be frozen for storage, later thawed and optionally diluted with a suitable diluent. Alternatively, the vector may be prepared as a composition which is suitable for delivery to a patient without proceeding through the freezing and thawing steps.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

VI. Other Vector

Provided herein is a vector comprising an expression cassette as described herein. In certain embodiments, the expression cassette comprises a nucleic acid sequence encoding a functional human methyl-CpG binding protein 2 (hMECP2) under control of regulatory sequences which direct the hMECP2 expression. In certain embodiments, the hMECP2-coding sequence encodes a hMECP sequence of [SEQ ID NO: 2; MAAAAAAAPSGGGGGGEEERLEEKSEDQDLQGLKDKPLKFKKVKKDKKEEKEGK HEPVQPSAHHSAEPAEAGKAETSEGSGSAPAVPEASASPKQRRSIIRDRGPMYDDPT LPEGWTRKLKQRKSGRSAGKYDVYLINPQGKAFRSKVELIAYFEKVGDTSLDPNDF DFTVTGRGSPSRREQKPPKKPKSPKAPGTGRGRGRPKGSGTTRPKAATSEGVQVKR VLEKSPGKLLVKMPFQTSPGGKAEGGGATTSTQVMVIKRPGRKRKAEADPQAIPKK RGRKPGSVVAAAAAEAKKKAVKESSIRSVQETVLPIKKRKTRETVSIEVKEVVKPLL VSTLGEKSGKGLKTCKSPGRKSKES SPKGRSSSASSPPKKEHHHHHHHSESPKAPVP LLPPLPPPPPEPESSEDPTSPPEPQDLSSSVCKEEKMPRGGSLESDGCPKEPAKTQPAV ATAATAAEKYKHRGEGERKDIVSSSMPRPNREEPVDSRTPVTERVS] is at least about 95% identical to SEQ ID NO: 3 and encodes a protein.

In certain embodiments, the vector is a viral vector selected from a recombinant parvovirus, a recombinant lentivirus, a recombinant retrovirus, or a recombinant adenovirus; or a non-viral vector selected from naked DNA, naked RNA, an inorganic particle, a lipid particle, a polymer-based vector, or a chitosan-based formulation. The selected vector may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

VII. Compositions

Provided herein is a composition comprising an rAAV or a vector as described herein and an aqueous suspension media. In certain embodiments, the suspension is formulated for intravenous delivery, intrathecal administration, or intracerebroventricular administration.

Provided herein are compositions containing at least one rAAV stock and an optional carrier, excipient and/or preservative. An rAAV stock refers to a plurality of rAAV vectors which are the same, e.g., such as in the amounts described below in the discussion of concentrations and dosage units.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Deliver vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In certain embodiments, the composition containing the rAAV.hMECP2 is delivered at a pH in the range of 6.8 to 8, or 7.2 to 7.8, or 7.5 to 8. For intrathecal delivery, a pH above 7.5 may be desired, e.g., 7.5 to 8, or 7.8.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. The aqueous solution may further contain Kolliphor® P188, a poloxamer which is commercially available from BASF which was formerly sold under the trade name Lutrol® F68. The aqueous solution may have a pH of 7.2.

In another embodiment, the formulation may contain a buffered saline aqueous solution comprising 1 mM Sodium Phosphate (Na₃PO₄), 150 mM sodium chloride (NaCl), 3 mM potassium chloride (KCl), 1.4 mM calcium chloride (CaCl₂)), 0.8 mM magnesium chloride (MgCl₂), and 0.001% poloxamer (e.g., Kolliphor®) 188, pH 7.2. See, e.g., harvardapparatus.com/harvard-apparatus-perfusion-fluid.html. In certain embodiments, Harvard's buffer is preferred due to better pH stability observed with Harvard's buffer.

In certain embodiments, the formulation buffer is artificial CSF with Pluronic F68. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In one example, the composition is formulated for intrathecal delivery. In one example, the composition is formulated for intravenous (iv) delivery.

VIII. Methods

Provided herein is a method of treating Rett Syndrome, comprising administrating an effective amount of an rAAV or a vector as described herein to a subject in need thereof.

In certain embodiments, an “effective amount” herein is the amount which achieves amelioration of RTT symptoms and/or delayed RTT progression.

The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. Conventional and pharmaceutically acceptable routes of administration include, but are not limited to, direct delivery to a desired organ (e.g., brain, CSF, the liver (optionally via the hepatic artery), lung, heart, eye, kidney,), oral, inhalation, intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired.

Dosages of the viral vector (for example, rAAV) depend primarily on factors such as the condition being treated, the age, weight and health of the patient, and can thus vary among patients. For example, a therapeutically effective human dosage of the viral vector is generally in the range of from about 25 to about 1000 microliters to about 100 mL of solution containing concentrations of from about 1×10⁹ to 1×10¹⁶ vector genome copies. In certain embodiments, a volume of about 1 mL to about 15 mL, or about 2.5 mL to about 10 mL, or about 5 mL suspension is delivered. In certain embodiments, a volume of about 1, about 2, about 3, about 4, about 5, about 6, about 7, about 8, about 9, about 10, about 11, about 12, about 13, about 14, or about 15 mL suspension is delivered. In certain embodiments, a dose of about 8.9×10¹² to 2.7×10¹⁴ GC total is administered in this volume. In certain embodiments, a dose of about 1.1×10¹⁰ GC/g brain mass to about 3.3×10¹¹ GC/g brain mass is administered in this volume. In certain embodiments, a dose of about 3.0×10⁹, about 4.0×10⁹, about 5.0×10⁹, about 6.0×10⁹, about 7.0×10⁹, about 8.0×10⁹, about 9.0×10⁹, about 1.0×10¹⁰, about 1.1×10¹⁰, about 1.5×10¹⁰, about 2.0×10¹⁰, about 2.5×10¹⁰, about 3.0×10¹⁰, about 3.3×10¹⁰, about 3.5×10¹⁰, about 4.0×10¹⁰, about 4.5×10¹⁰, about 5.0×10¹⁰, about 5.5×10¹⁰, about 6.0×10¹⁰, about 6.5×10¹⁰, about 7.0×10¹⁰, about 7.5×10¹⁰, about 8.0×10¹⁰, about 8.5×10¹⁰, about 9.0×10¹⁰, about 9.5×10¹⁰, about 1.0×10¹¹, about 1.1×10¹¹, about 1.5×10¹¹, about 2.0×10¹¹, about 2.5×10¹¹, about 3.0×10¹¹, about 3.3×10¹¹, about 3.5×10¹¹, about 4.0×10¹¹, about 4.5×10¹¹, about 5.0×10¹¹, about 5.5×10¹¹, about 6.0×10¹¹, about 6.5×10¹¹, about 7.0×10¹¹, about 7.5×10¹¹, about 8.0×10¹¹, about 8.5×10¹¹, about 9.0×10¹¹ GC per gram brain mass is administered in this volume.

The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

The replication-defective virus compositions can be formulated in dosage units to contain an amount of replication-defective virus that is in the range of about 1.0×10⁹ GC to about 1.0×10¹⁶ GC (to treat an subject) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰ 3×10¹⁰ 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range.

In one embodiment, for human application the dose can range from 1×10¹⁰ to about 1×10¹⁵ GC per kg body weight including all integers or fractional amounts within the range. In one embodiment, the effective amount of the vector is about 1×10⁹, 2×10⁹, 3×10⁹, 4×10⁹, 5×10⁹, 6×10⁹, 7×10⁹, 8×10⁹, or 9×10⁹ GC per kg body weight including all integers or fractional amounts within the range. In another embodiment, the effective amount of the vector is about 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per kg body weight including all integers or fractional amounts within the range. In another embodiment, the effective amount of the vector is about 1×10¹¹, 2×10¹¹, 3×10¹¹, 4×10¹¹, 5×10¹¹, 6×10¹¹, 7×10¹¹, 8×10¹¹, or 9×10¹¹ GC per kg body weight including all integers or fractional amounts within the range. In another embodiment, the effective amount of the vector is about 1×10¹², 2×10¹², 3×10¹² 4×10¹², 5×10¹², 6×10¹², 7×10¹², 8×10¹², or 9×10¹² GC per kg body weight including all integers or fractional amounts within the range. In another embodiment, the effective amount of the vector is about 1×10¹³, 2×10¹³, 3×10¹³, 4×10¹³, 5×10¹³, 6×10¹³, 7×10¹³, 8×10¹³, or 9×10¹³ GC per kg body weight including all integers or fractional amounts within the range. In another embodiment, the effective amount of the vector is about 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per kg body weight including all integers or fractional amounts within the range. In another embodiment, the effective amount of the vector is about 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per kg body weight including all integers or fractional amounts within the range.

In one embodiment, for human application the dose can range from 1×10¹⁰ to about 1×10¹⁵ GC per gram (g) brain mass including all integers or fractional amounts within the range. In one embodiment, the effective amount of the vector is about 1×10⁹, 2×10⁹, 3×10⁹, 4×10⁹, 5×10⁹, 6×10⁹, 7×10⁹, 8×10⁹, or 9×10⁹ GC per gram (g) brain mass including all integers or fractional amounts within the range. In another embodiment, the effective amount of the vector is about 1×10¹⁰, 2×10¹⁰ 3×10¹⁰ 4×10¹⁰, 5×10¹⁰, 6×10¹⁰, 7×10¹⁰, 8×10¹⁰, or 9×10¹⁰ GC per gram (g) brain mass including all integers or fractional amounts within the range. In another embodiment, the effective amount of the vector is about 1×10¹¹, 2×10¹¹, 3×10¹¹, 4×10¹¹, 5×10¹¹, 6×10¹¹, 7×10¹¹, 8×10¹¹, or 9×10¹¹ GC per gram (g) brain mass including all integers or fractional amounts within the range. In another embodiment, the effective amount of the vector is about 1×10¹², 2×10¹², 3×10¹², 4×10¹², 5×10¹², 6×10¹², 7×10¹², 8×10¹², or 9×10¹² GC per gram (g) brain mass including all integers or fractional amounts within the range. In another embodiment, the effective amount of the vector is about 1×10¹³, 2×10¹³, 3×10¹³, 4×10¹³, 5×10¹³, 6×10¹³, 7×10¹³, 8×10¹³, or 9×10¹¹ GC per gram (g) brain mass including all integers or fractional amounts within the range. In another embodiment, the effective amount of the vector is about 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per gram (g) brain mass including all integers or fractional amounts within the range. In another embodiment, the effective amount of the vector is about 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per gram (g) brain mass including all integers or fractional amounts within the range.

The above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 pt. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 1×10¹⁰ GC/g brain mass to about 3×10¹¹ GC/g brain mass. In certain embodiments, the dose may be in the range of about 1×10¹⁰ GC/g brain mass to about 2.5×10¹¹ GC/g brain mass. In certain embodiments, the dose may be in the range of about 5×10¹⁰ GC/g brain mass.

In one embodiment, the viral constructs may be delivered in doses of from at least about least 1×10⁹ GC to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage may be adjusted to balance the therapeutic benefit against any side effects and such dosages may vary depending upon the therapeutic application for which the recombinant vector is employed.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other route of delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna. In certain embodiment, a rAAV, vector, or composition as described herein is administrated to a subject in need via the intrathecal administration. In certain embodiments, the intrathecal administration is performed as described in US Patent Publication No. 2018-0339065 A1, published Nov. 29, 2019, which is incorporated herein by reference in its entirety.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In certain embodiments, treatment of the composition described herein has minimal to mild asymptomatic degeneration of DRG sensory neurons in animals and/or in human patients, well-tolerated with respect to sensory nerve toxicity and subclinical sensory neuron lesions.

IX. Apparatus and Method for Delivery of a Pharmaceutical Composition into Cerebrospinal Fluid

In one aspect, the vectors provided herein may be administered intrathecally via the method and/or the device provided in this section and described in WO 2018/160582, which is incorporated by reference herein. Alternatively, other devices and methods may be selected. In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis. In certain embodiments, the apparatus is described in US Patent Publication No. 2018-0339065 A1, published Nov. 29, 2019, which is incorporated herein by reference in its entirety.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “expression” is used herein in its broadest meaning and comprises the production of RNA or of RNA and protein. With respect to RNA, the term “expression” or “translation” relates in particular to the production of peptides or proteins. Expression may be transient or may be stable.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

As described above, the term “about” when used to modify a numerical value means a variation of ±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLES

The following examples are illustrative only and are not intended to limit the present invention.

A gene therapy was developed to restore MECP2 expression which has been identified as causative gene defect. An MECP2 expression cassette is transferred into brain neurons using AAV gene therapy technology. The injection of the therapeutic article is delivered via cerebrospinal fluid (CSF), using the cisterna magna as access point, or intravenously. A MECP2 expression cassette highly optimized for expression in humans was used in combination with an AAV capsid with highly improved central nervous system (CNS) transduction.

### Example 1—hSyn-MECP2co Vector

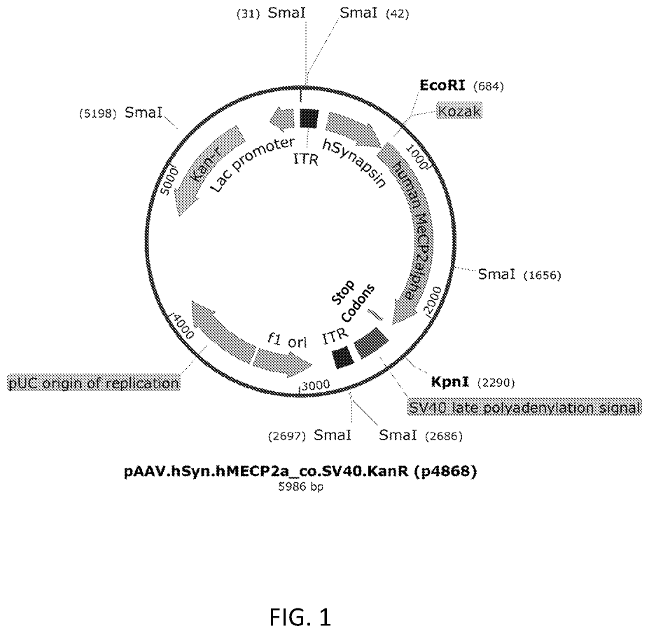

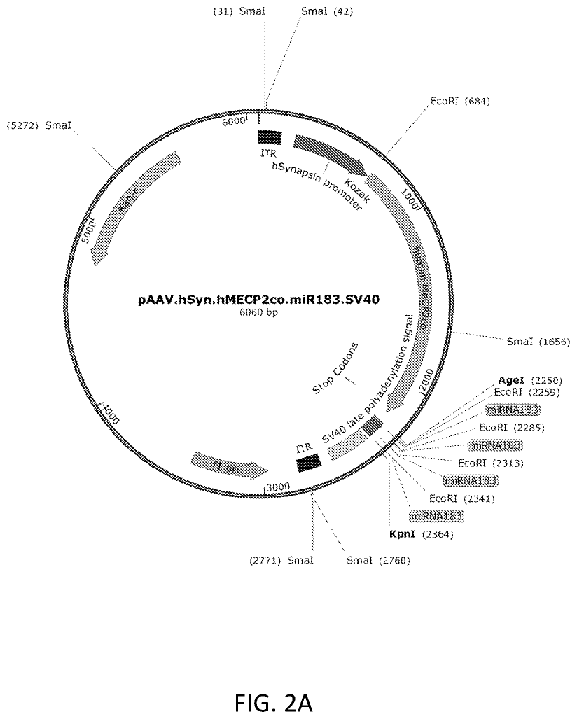

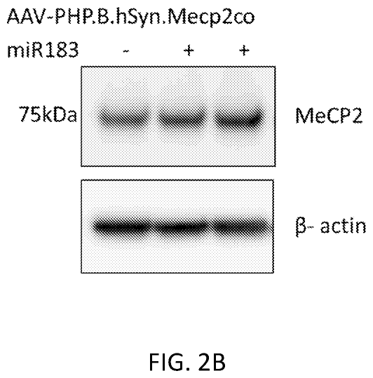

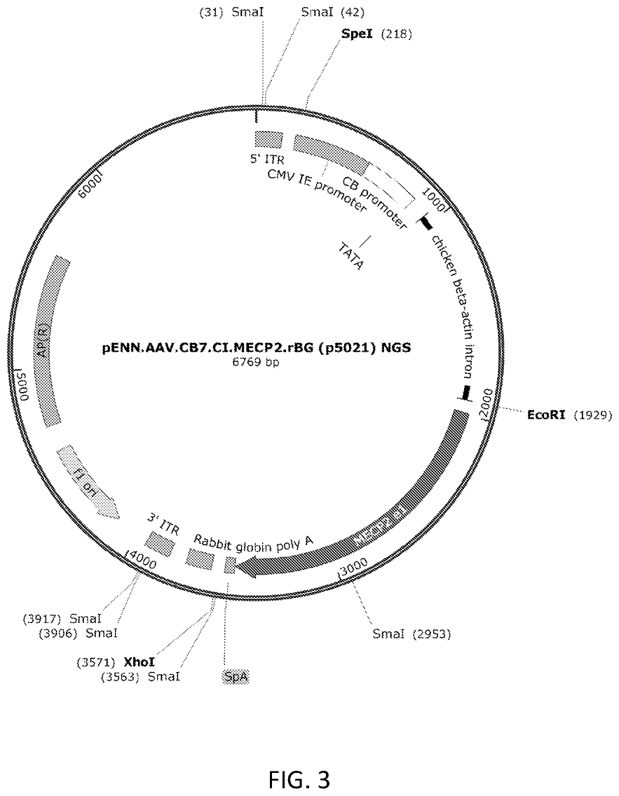

Plasmids. The amino acid sequence for the main human isoform of MECP2 (Methyl-CpG-binding protein 2, isoform alpha, UniProt ID P51608-2) was reverse translated into a DNA sequence and then further engineered. The engineered MECP2 sequence (SEQ ID NO 3, i.e., nt 696 to nt 2195 of SEQ ID NO: 1 and nt 696 to nt 2195 of SEQ ID NO: 6, which is also termed as hMECP2co or MECP2co as used herein) was cloned into an AAV expression plasmid under the control of the human synapsin promoter (Thiel, G., Greengard, P. & Südhof, T. C. Characterization of tissue-specific transcription by the human synapsin I gene promoter. Proceedings of the National Academy of Sciences 88, 3431-3435, doi:10.1073/pnas.88.8.3431 (1991)). The MECP2 coding sequence is preceded by a Kozak sequence, followed by the SV40 poly A sequence and framed by AAV2 inverted terminal repeats (ITR) (FIG. 1; SEQ ID NO: 1). Alternatively, in some experiments, a rabbit globulin poly A sequence was used (FIG. 3; SEQ ID NO: 4). To suppress expression in dorsal root ganglia (DRG), in some experiments the above plasmid was modified to contain four repeats of the miR183 binding site (agtgaattctaccagtgccata, SEQ ID NO: 7) directly after the MCEP2 coding sequence and before the SV40 poly A sequence (FIG. 2A; SEQ ID NO: 6). AAV MECP2 vector was produced at the University of Pennsylvania Vector Core, using either as capsid PHP.B (Deverman, B. E. et al. Cre-dependent selection yields AAV variants for widespread gene transfer to the adult brain. Nat Biotechnol 34, 204-209, doi:10.1038/nbt.3440 (2016)) or AAV9 or AAVhu68 (Hinderer, C. et al. Severe Toxicity in Nonhuman Primates and Piglets Following High-Dose Intravenous Administration of an Adeno-Associated Virus Vector Expressing Human SMN. Human Gene Therapy 29, 285-298, doi:10.1089/hum.2018.015 (2018)) for mouse studies or studies with non-human primates.

### Example 2—Mouse Study

1. Materials and Methods

Mouse Studies. Mecp2-ko mice (ko mice) were obtained from Jackson Laboratories (B6.129P2(C)-Mecp2tm1.1Bird/J, strain #003890) and crossed heterozygous female ko mice with wildtype (wt) C57Bl6 males to obtain male Mecp2-ko mice (Hemizygote, HEMI, also noted as ko mice) and male wt litter mates. Mice received AAV.MECP2 vector or vehicle control (sterile phospho-buffered saline, PBS) at 18-21 days of age by retro-orbital injections of 1×10¹¹ to 5×10¹¹ genome copies (gc) in a total volume of 100 ul per mouse. Mouse were housed mixed in regard of genotype and injection product, weighed and observed at least twice a week, and aged to three months. Most Mecp2-ko mice that had received vehicle control died or reached a humane endpoint for euthanasia before reaching the 3-month timepoint whereas AAV MECP2-treated and wt mice survived and were fit for undergoing a battery of behavior testing.

Western Blotting and Tissue Staining. After euthanasia, one cortical hemisphere was flash frozen and subsequently protein lysates were generated using RIPA buffer. Western blotting was carried out with antibodies against murine and human MECP2 (PA1-887, Thermo Fisher). The other brain half was fixed overnight in formalin, embedded in paraffin and thin sections were processed for immunofluorescence staining with the same antibodies. Alternatively, tissue was stained with H&E for pathology review. Decalcified spinal cord cross sections were also stained with Luxol Fast Blue stain (Sigma-Aldrich, Inc).

Behavior Testing. Mice underwent one test per day. Testing time of day, operator and environment was kept the same (60 dB white noise background and 1000 lumens incandescent indirect lighting). Mice were habituated in their home cages before each test for 30 minutes. For the Open Field Assay, a new cage with minimal amount of bedding was placed into an infrared beam array (MedAssociates, Inc.). A single mouse was added in the middle of the cage and the number of beam brakes over the next 30 minutes was automatically recorded, separated into beam brakes close to the ground (general activity) and 3 inches above ground (rearing activity). For the Elevated Zero Maze (EZM, Stoelting Co.), an elevated circular platform with two opposite enclosed quadrants and two open was used to allow uninterrupted exploration. A single mouse was place in the middle of an open quadrant and movement was video-recorded for 15 minutes. For the Y-Maze (Stoelting Co.), an enclosed platform containing three identical arms in the shape of a Y was used. A single mouse was placed in the arm closest to the operator and its movements were video-recorded for 5 minutes. For the Marble Burying Assay, a fresh cage was filled with 3 inches of AlphaDri bedding (Shepherd Specialty Papers) and gently compacted down. 12 solid-blue marbles were spaced equally onto the bedding and a single mouse placed in the middle of the cage. The number of marbles that were at least half covered by bedding was recorded after 30 minutes. For the rotarod assay, an accelerating rotating beam was used (Ugo Basile SA). On the first day, mice were accustomed to the rotating beam on its lowest rotational speed (4 rpm (revolutions per minute)) for three session of 5 minutes each. For testing, mice were placed on the beam slowly rotating at 4 rpm. The speed was increased over 5 minutes to 40 rpm final rotational speed. The latency to fall was recorded for each mouse. After 15 minutes of resting, the same experiment was repeated twice. For some experiments, rotator testing was done on three consecutive days with three sessions per day.

Data analysis. Data was graphed and analyzed using GraphPad Prims software. Video files were recorded in mp4 format at 20 fps and analyzed using EthoVision XT software (version 14, Noldus Information Technology).

2. Pharmaceutical Efficacy

An AAV vector for MECP2 gene therapy (AAV.hMECP2co) was developed. The DNA sequences of MECP2 were engineered for expression of hMECP2 of the amino acid sequence of SEQ ID NO:2. Expression is driven by the human synapsin promoter, which has extensively documented to selectively express in CNS neurons. Result shows that the AAV-PHP.B.hSyn-MECP2co vector carrying the MECP coding sequence of SEQ ID NO: 3 transduced a very high fraction of mouse CNS neurons and facilitated robust and wide-spread MECP2 protein expression. It is also shown that treatment of juvenile Mecp2-ko mice by intravenous (IV, or iv) injection with this vector overcame early mortality and significantly improved behavior outcome measures. Dose-optimization allowed achieving behavior correction closely resembling wildtype performance.

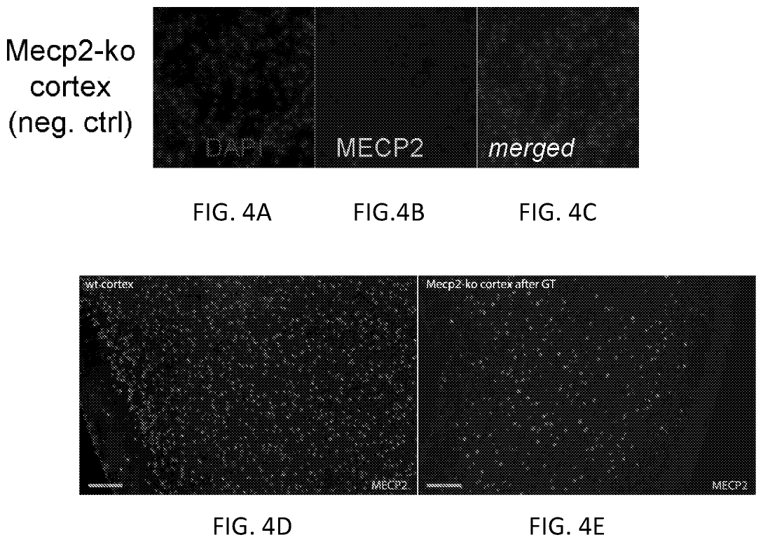

A successful expression of MECP2 via the AAV-PHP.B.hSyn.MECP2co vector was observed in mouse brain. The AAV-PHP.B.hSyn.MECP2co vector was injected to Mecp2-ko mice intravenously (iv) for maximal brain transduction. Mouse brains were then harvested and processed for immunofluorescence staining using anti-MECP2 antibody as described in the Materials and Methods. Representative images are provided in FIGS. 4A-4E and the result shows that a widespread expression of hMECP2 was achieved in the cortex of the Mecp2-ko mice treated by the AAV-PHP.B.hSyn.MECP2co vector (FIG. 4E) but not in the untreated ko mice served as negative controls (FIGS. 4B and 4C). Also, the hMECP2 expression level in the treated Mecp2-ko mice was similar to that observed in wildtype cortex (FIG. 4D, positive control). Additionally, successful expression of MECP2 via AAV-PHP.B.hSyn.MECP2co.miR183 was observed in mouse brain (FIG. 2B). Thereby, MECP2 expression in mouse brain is not affected with addition of miR183 targeting sequence to an expression cassette.

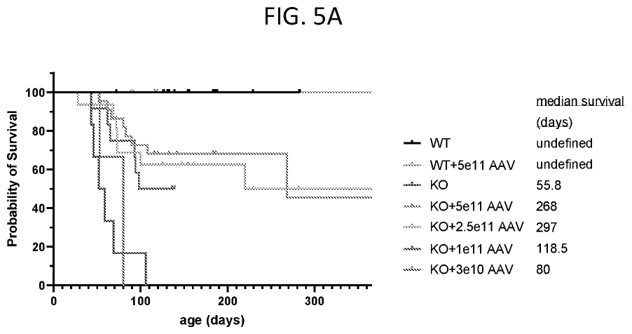

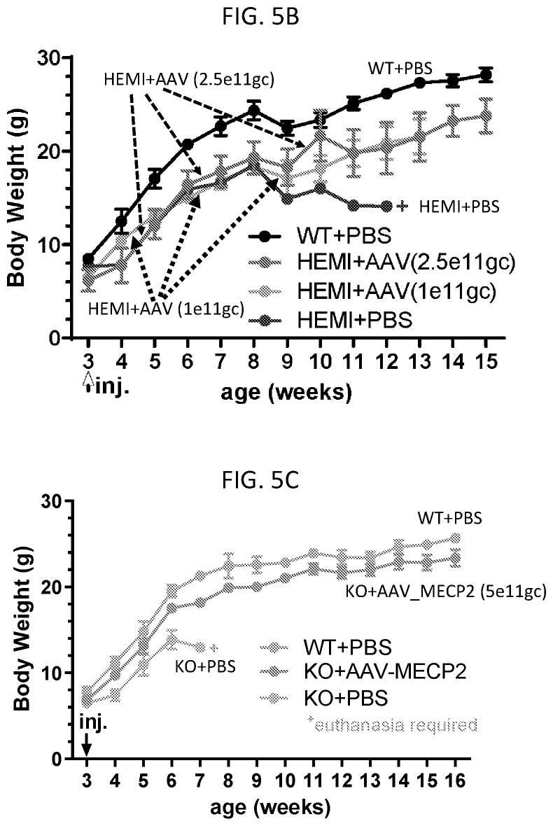

Therapeutic efficacy in juvenile Mecp2-ko mice was further investigated. The AAV-PHP.B.hSyn.MECP2co vector led to an increased lifespan at a lower dose compared to a dose of 5×10¹¹ GC/juvenile male mouse which was established as the initial dose with therapeutic efficacy. Briefly, male Mecp2-ko mice (i.e., HEMI or KO or ko) were injected i.v. with four doses of the AAV-PHP.B.hSyn.MECP2co vector (3×10¹⁰ GC/mouse, noted as 3e10 AAV; 1×10¹¹ GC/mouse, noted as 1e11 AAV; and 2.5×10¹¹ GC/mouse, noted as 2.5e11 AAV, and 5×10¹¹ GC/mouse, noted as Sell AAV) in FIG. 5A, as described in the Materials and Methods. Male wild type littermates injected with phosphate-buffered saline (PBS) severed as a positive control (noted as PBS, WT or WT+PBS in figures), while male Mecp2-ko mice treated with PBS severed as a negative control (noted as KO, ko+PBS or KO+PBS or HEMI+PBS in figures). Percentages of survived mice in the tested groups were plotted in FIG. 5A in Kaplan-Meier survival plot, showing that highest three doses (1×10¹¹, 2.5×10¹¹ and 5×10¹¹ GC/mouse) significantly improved overall survival of the Mecp2-ko mice compared to the negative control; and all of the ko mice treated with 1×10¹¹ GC of the vector as well as the wild type (wt) littermates survived at the end of the observation period (at least until 16 weeks of age as shown in FIG. 5A). All of the KO mice treated with lowest dose of 3×10¹¹ GC/mouse did not survive past day 80 of the observation period. Body weight of the tested mice were also monitored (FIGS. 5B and 5C). The ko mice treated with three doses (1×10¹¹, 2.5×10¹¹ and 5×10¹¹ GC/mouse) of the vector showed a steady increase in body weights, while starting from 6-9 weeks of age, the PBS-treated ko mice gradually lost instead of gained weights. The PBS treated wild type mice displayed a bodyweight curve similar to those of the treated ko mice but with slightly heavier weights.

Furthermore, the therapeutic efficacy in neonatal Mecp2-ko mice was further investigated with additional range of doses administered via ICV: 6×10⁹ GC/mouse to 6×10¹⁰ GC/mouse (4 doses).

DRG de-targeting is incorporated in MECP2 expression cassette to allow for higher dose of the vector administrated. The therapeutic efficacy of AAVhu68.hSyn.MECP2co.miR183, comprising an addition of miR183 sequence for DRG de-targeting, was examined. The vector was delivered to juvenile subject at a dose of 2.3×10¹¹ GC/mouse via ICM injection.

A dose-dependent behavior correction was observed after the MECP2 gene therapy. Behavior tests, including Open Field Assay, Elevated Zero Maze, Y-Maze, Marble Burying Assay, and rotarod assay, were tested on male Mecp2-ko mice (i.e., HEMI) injected iv with three different doses of the AAV-PHP.B.hSyn.MECP2co vector (1×10¹¹ GC/mouse, also noted as HEMI+AAV (1e11gc); 2.5×10¹¹ GC/mouse, noted as HEMI+AAV (2.5e11gc); and 5×10¹¹ GC/mouse, noted as HEMI+AAV (2.5e11gc)), phosphate-buffered saline (PBS) treated male wild type littermates (positive control, labelled as PBS or WT+PBS in figures), and PBS treated ko mice (negative control, noted as HEMI+PBS).

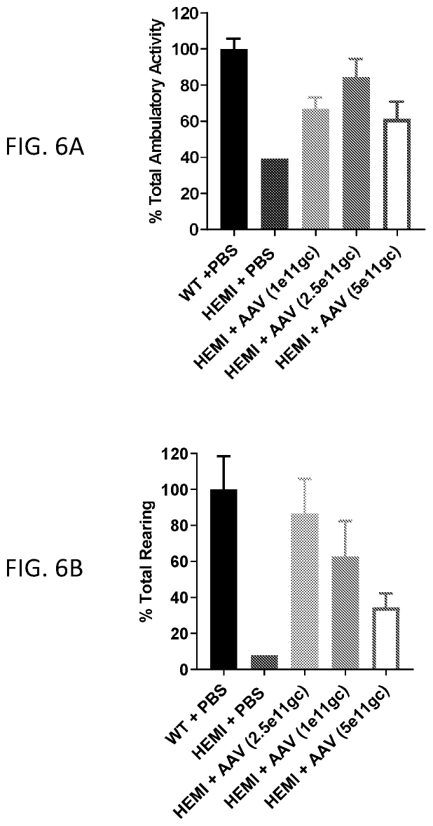

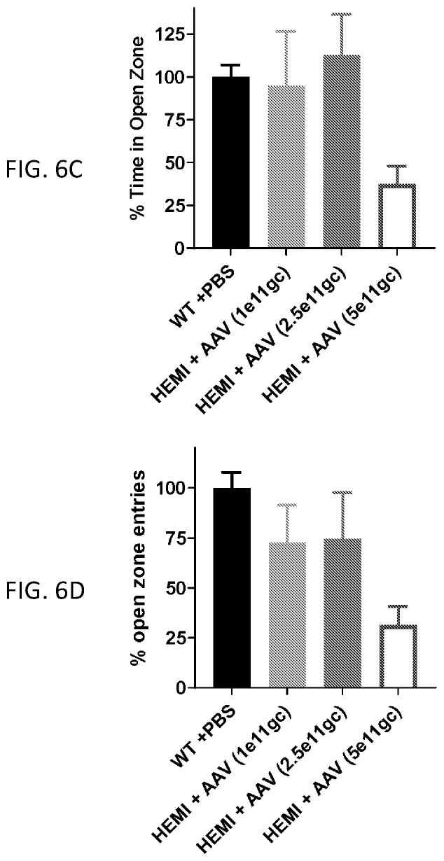

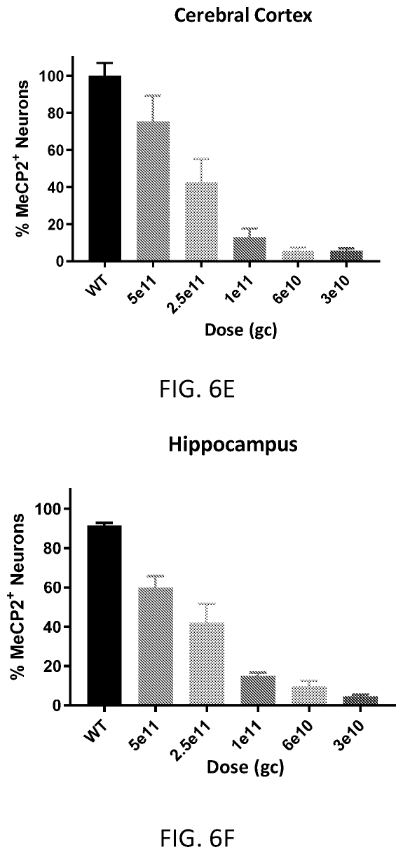

In the Open Field Assay, the averaged percentage of the total ambulatory activity or the rearing activity acquired from the PBS-treated WT was set as 100% and the readings from the rest groups were then calculated accordingly and presented in FIGS. 6A and 6B. The PBS treated ko mice showed an about 40% ambulatory activity and a less than 10% rearing activity. Treatment via 1×10¹¹ GC of the vector increased the ambulatory activity to about 67%, and 2.5×10¹¹ GC of the vector further improved the percentage to about 85%. The rearing activity was increased by 1×10¹¹ GC or 2.5×10¹¹ GC of the vector up to about 85% or about 60% respectively. Yet, the highest dose tested (5×10¹¹ GC/mouse) demonstrated an about 60% ambulatory activity and an about 32% rearing activity, both of which are still higher than that of PBS treated ko mice.

Time spent in open zone as well as numbers of open zone entries in the Elevated Zero Maze were monitored and the results are presented in FIGS. 6C and 6D. In both figures, the average readings of the wt mice were set as 100% and relative percentages of the rest groups were calculated accordingly. For both assessments performed, ko mice treated with 1×10¹¹ GC or 2.5×10¹¹ GC of the vector showed a percentage comparable to the wt control. Decreases in time in Open Zone as well as in open zone entries were observed in ko mice treated with 5×10¹¹ GC vector. Further, the ko mice administrated with only PBS moved very little and did not leave the open zone they were initially placed in, thus it was impossible to assess their preference for the open or closed zone. Additionally, a correlation analysis was performed of behavior outcome and number of the neurons transduced in the mouse brain post treatment. Triple-stained immunofluorescence images (DAPI for nuclei, MECP2, and NeuN for Neurons) were used to semi-automatically quantify the number of neurons expressing MECP2 in the Mecp2-ko cerebral cortex (FIG. 6E) and hippocampus (FIG. 6F) after injections at different doses. The obtained data was graphed as % MECP2+/NeuN+ cells. Animals treated with the doses of 6×10¹⁰ GC/mouse or below (3×10¹⁰ GC/mouse) did not survive long enough and/or did not have enough mobility to proceed with behavior test. In correlation only small number of neurons were observed to stain positive for expression of MECP2 (MECP2+). In cerebral cortex, 5.7% and 5.8% were MECP2+ neurons, respectively at doses 3×10¹⁰ and 6×10¹⁰ GC/mouse. In hippocampus, 5.1% and 10.8% were MECP2+ neurons, respectively at doses 3×10¹⁰ and 6×10¹⁰ GC/mouse. In cerebral cortex, 12.9%, 42.6%, and 75.3% were MECP2+ neurons, respectively at doses 1×10¹¹, 2.5×10¹¹, and 5×10¹¹ GC/mouse. In hippocampus, 16.3%, 46.0% and 65.4% were MECP2+ neurons, respectively at doses 1×10¹¹, 2.5×10¹¹, and 5×10¹¹ GC/mouse. Therefore, 1×10¹¹ GC/mouse was deemed the minimal effective dose.

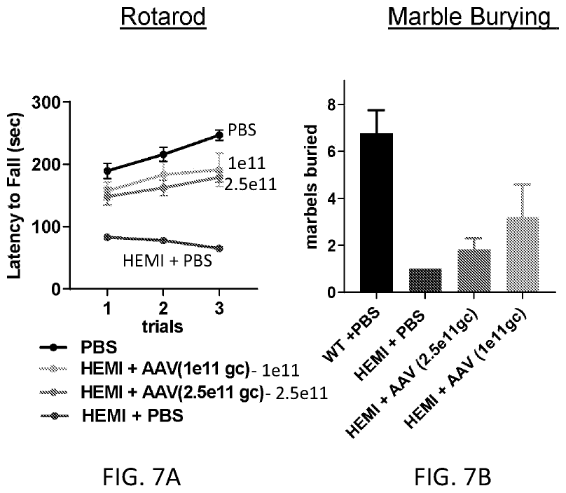

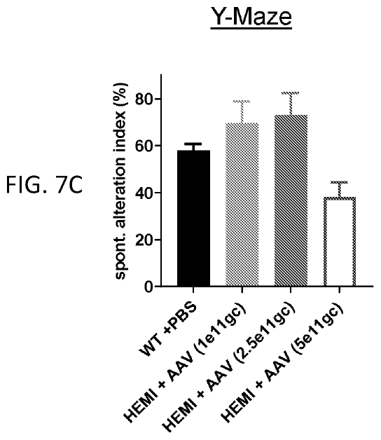

Motor coordination of the mice was also evaluated via rotarod test. Latency to fall measured by seconds of a mouse staying on a rotarod is shown in FIG. 7A. Compared to the PBS treated ko mice, the ko mice treated with 1×10¹¹ GC or 2.5×10¹¹ GC of the vector showed a significant improvement on staying on the rotarod and avoiding falling. Additionally, three trials were performed for each mouse. No improvement while repeating the rotarod test was observed in PBS treated ko mice. However, an increase in time on the rotarod was observed from PBS-treated wt mice as well as the vector treated ko mice.

Behavior trait of the mice was further tested via Marble Burying assay. An about 7 out of 12 marbles were at least half covered by bedding by the wt mice, while PBS treated ko mice only buried 1. An about 2, or an about 3 marbles were buried by ko mice treated with 1×10¹¹ GC or 2.5×10¹¹ GC of the vector, respectively, demonstrating a behavior correction toward wt phenotype.

In a Y Maze, a mouse typically prefers to investigate a new arm of the maze rather than returning to one that was previously visited. Thus, Y Maze spontaneous alternation index may serve as an indicator for willingness to explore new environments. As shown in FIG. 7C, treatment with 5×10¹¹ GC of the vector brought the index up to about 35%, while the indexes of ko mice treated with 1×10¹¹ GC or 2.5×10¹¹ GC of the vector were about 70% which is similar to if not higher than the wt one (about 58%). Ko mice receiving only PBS moved very little and did not reach the threshold of at least 9 decisions per 5 minute interval, thus it was not possible to assess their performance in the Y Maze.

The table below further provides pharmaceutical efficacy of the AAV-PHP.B.hSyn.MECP2co vector in juvenile ko mice shown as a more than 100-day survival or as comparisons to a wild-type (wt) control. These results indicate that behavior correction outcome depends on AAV.hMECP2co dose.

| | Note (e.g., | | | |
|---|---|---|---|---|
| | measurements | 5 × 10¹¹ | 2.5 × 10¹¹ | 1 × 10¹¹ |
| Dose | taken) | GC/mouse | GC/mouse | GC/mouse |
| Survival | Currently | >250 days | >155 days | >115 days |
| | on study | | | |
| Weight | 3 to 16 | normal | Slightly | Slightly |
| | weeks age | | leaner | leaner |
| Rotarod | Time to fall | 71% of wt | | |
| Open field | Beams broken | 51% of wt | 84% of wt | 67% of wt |
| Elevated maze | Time in | 37% of wt | 112% of wt | 94% of wt |
| | open zone | | | |
| Y-Maze | Spontaneous | 66% of wt | 126% of wt | 121% of wt |
| | alterations | | | |
| Marble | Marbles | (10% | 27% of wt | 47% of wt |
| burying | buried | of wt) | | |

In summary, this therapeutic efficacy study in juvenile mice showed that treatment with 1×10¹¹, 2.5×10¹¹ and 5×10¹¹ GC/mouse of AAV-PHP.B.hSyn-hMECP2co led to an increased life span and prevented onset of typical symptoms in mice, and lowering of treatment dose improved outcome of behavior correction.

AAV.hMECP2co vectors utilizing promoters with proven expression efficacy in NHP (for example, CBA or UbC promoter) but the same hMECP2co nucleic acid sequence are also tested in juvenile mice and shows pharmaceutical efficacy.

Dose-dependency is established between number of neurons transduced and behavior correction. The achieved per-cell expression levels are also compared to that of wt mouse and human brain.

3. Safety/Toxicity

Juvenile or adult wild type C57Bl6 mice were injected with the AAV-PHP.B.hSyn-hMECP2co vector i.v. at various doses in order to evaluate safety or toxicity of the vector.

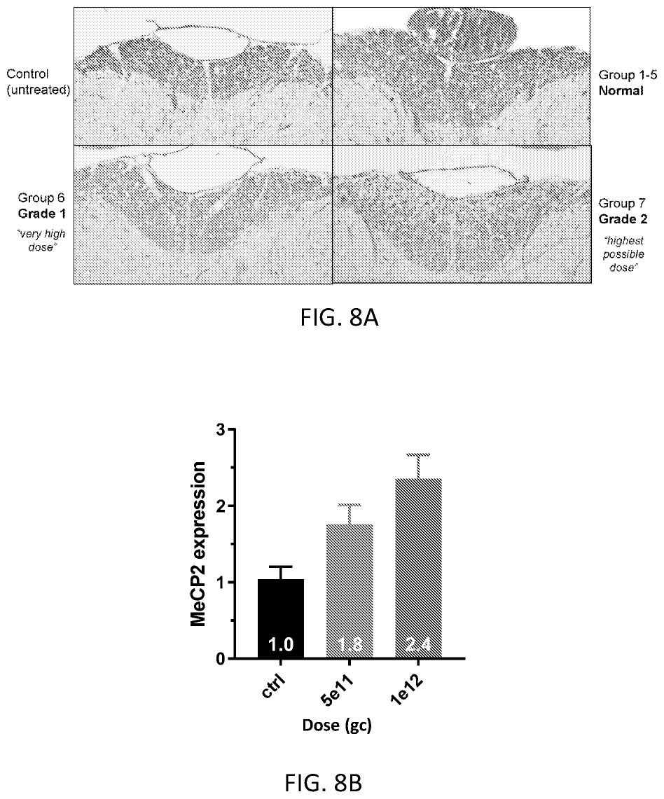

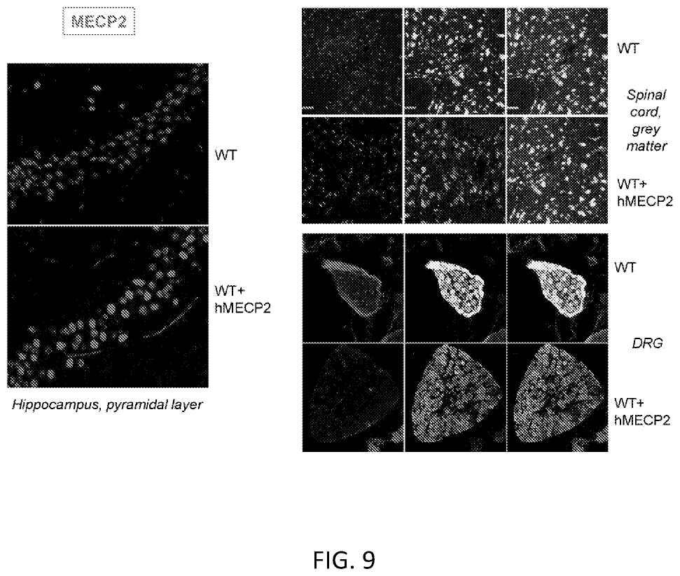

Treatment of juvenile wt mice with an increasing dose of AAV-hSyn-hMECP2 only adversely affected motor function and caused mild spinal axionopathy at very high dose (5×10¹² GC/mouse, which is 50 times of the lowest therapeutic dose, 1×10¹¹ GC/mouse). Juvenile wt mice were treated with the AAV-PHP.B.hSyn-hMECP2co vector i.v. at doses of 3×10⁹ GC/mouse (Group 1), 1×10¹⁰ GC/mouse (Group 2), 5×10¹⁰ GC/mouse (Group 3), 1×10¹¹ GC/mouse (Group 4), 5×10¹¹ GC/mouse (Group 5), 1×10¹² GC/mouse (Group 6) and 5×10¹² GC/mouse (Group 7). No morbidity or mortality was observed in the vector-treated juvenile wt mice; and expression of hMECP2 showed no toxicity effect at and under the dose of 5×10¹¹ GC/mouse (Groups 1-5). Overt symptoms (for example, reduce ability to move due to hind-leg effect) were only observed in highest treatment group (Group 7). Axonopathy in dorsal white matter were found in the mice treated with 1×10¹² GC/mouse and 5×10¹² GC/mouse of the vector. Axonopathy in dorsal white matter tracts and dorsal nerve roots were investigated. Cells and MECP2 positive cells in pyramidal layer of hippocampus, grey matter of spinal cord and dorsal root ganglion cells (DRG) were compared between the wild type mice with or without the vector treatment (FIG. 9). No loss of dorsal root ganglion cells was observed in all tested groups. However, Luxol Fast stain showed that a Grade 1, mild demyelination was detected in the mice treated with 1×10¹² GC vector, while a Grade 2, moderate demyelination was observed in the 5×10¹² GC group. No demyelination was found in lower dosage groups (Groups 1-5). See, FIG. 8A. Additionally performed Western blot analysis showed relative overexpression of MECP2 in wt brains after AAV vector injection at doses 5×10¹¹ (as noted Sell) and 1×10¹² (as noted 1e12) GC/mouse. Performed in parallel pathological analysis provided that neuronal pathology was observed to be normal at 5×10¹¹ GC/mouse dose (with 1.8-fold MECP2 expression compared to WT levels). However, a grade 1 axonopathy became apparent at 1×10¹² GC/mouse dose (with 2.4-fold MECP2 compared to WT levels).

Treatment of adult wt mice with a high dose of AAV-hSyn-hMECP2 adversely affected behavior outcome (decreased activity) but did not cause overt motor defects (at 4× of the lowest therapeutic dose). The AAV.hMECP2co vector was administrated i.v. to adult mice at a dose of 1×10¹² GC/mouse. This dose in adult mice is equivalent to 4×10¹¹ GC/juvenile mouse since body weight of an adult mice (about 25 g) is about 2.5 times of a juvenile (about 10 g). Therefore, this tested dose (1×10¹² GC/adult mouse) is believed to be 4 times of the lowest therapeutic dose which is 1×10¹¹ GC/juvenile mouse.

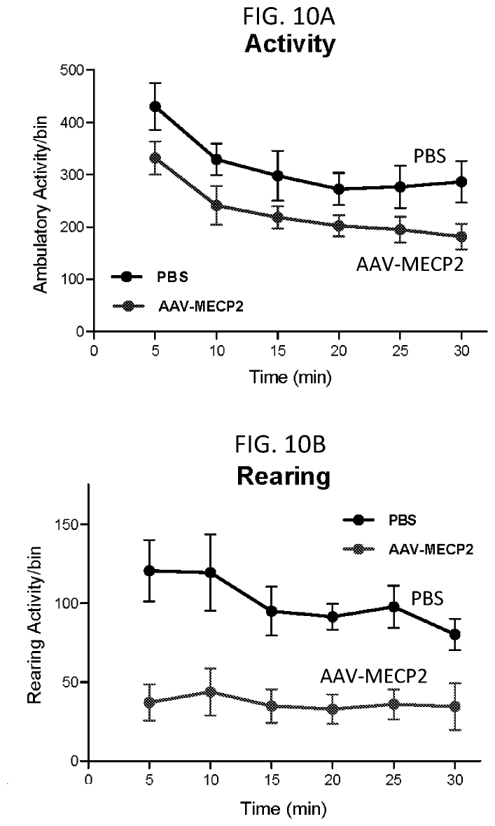

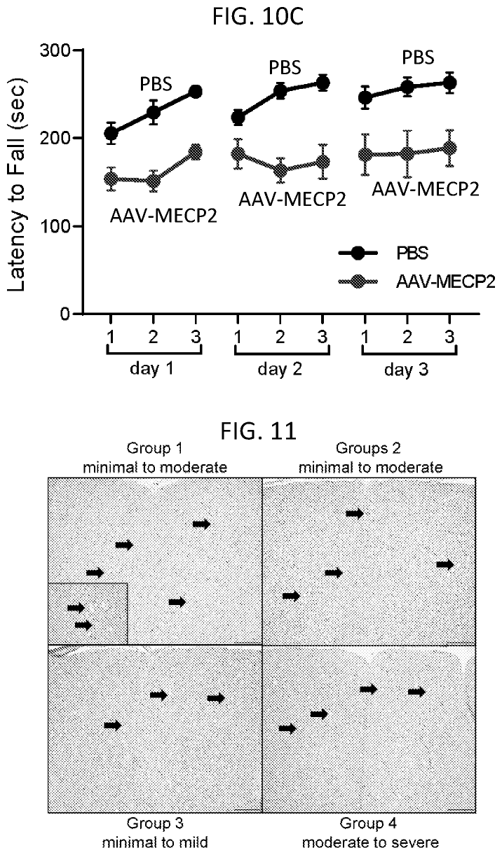

Open Field Assay was performed to evaluate activity level of the adult mice. It was showed that treatment via 1×10¹² GC/mouse of the vector led to an ambulatory/horizontal activity reduction by 28% (p<0.0001, ANOVA, FIG. 10A) and a rearing/vertical activity reduction by 64% (p<0.0001, ANOVA, FIG. 10B) compared to the control group treated by only PBS. Neuro-motor ability and coordination were investigated by rotarod tests. As shown in FIG. 10C, the vector-treated mice were more likely to fall from the rotarod compared to the PBS treated mice (p<0.001, 2-way ANOVA). However, this result may be confounded by the lower activity shown in FIGS. 10A and 10B. iv administration of the AAV.hMECP2co vector to adult wt mice at doses of 5×10¹⁰, 1×10¹¹, and 5×10¹¹ GC/mouse is also performed. The mice are then tested using the behavior assays as described above.

AAV.hMECP2co vectors utilizing promoters with proven expression efficacy in non-human primate (NHP) (for example, CBA or UbC promoter) but the same hMECP2co nucleic acid sequence are also tested in juvenile and adult mice for safety and toxicity as described herein.

### Example 3—Non-human Primate (NHP) Study

1. Materials and Methods

Non-human primate experiments. Non-human primates (NHPs) for the species Macaca mulatta (rhesus macaques) were obtained from Covance Research Products, Inc. Quarantine and animal husbandry was performed according to Gene Therapy Program SOPs. In the month before AAV vector administration and throughout the study body weight, temperature, respiratory rate and heart rate were periodically monitored, and blood and CSF samples were obtained. Whole blood was used for cell counts and differentials, and a clinical blood chemistry panel. CSF samples were used for blood cell counts and differentials, and total protein quantification. For AAV vector delivery into the CSF via puncture of the cisterna magna, anesthetized macaques were placed on a procedure table in the lateral decubitus position with the head flexed forward. Using aseptic technique, a 21-27 gauge, 1-1.5 inch Quincke spinal needle (Becton Dickinson) was advanced into the sub-occipital space until the flow of CSF was observed. The needle was directed at the wider superior gap of the cisterna magna to avoid blood contamination and potential brainstem injury. Correct placement of needle puncture was verified via myelography, using a fluoroscope. 1 mL of CSF was collected for baseline analysis, prior to dosing. After CSF collection, a leur access extension catheter was connected to the spinal needle to facilitate dosing of 1 ml Iohexol (Trade Name: Omnipaque 180 mg/mL, General Electric Healthcare) contrast media. After verifying needle placement, a syringe containing the test article (volume equivalent to 1 mL plus the volume of syringe and linker dead space) was connected to the flexible linker and injected over 30±5 seconds. The needle was removed, and direct pressure was applied to the puncture site. AAVhu68.hSyn.MECP2co vector has injected at a dose of 3×10¹², 1×10¹³ or 3×10¹³ GC/NHP while AAVhu68.MECP2 vector was injected at a dose of 2×10¹³ GC/NHP. At study days 0, 14, 18, 41 and the last study day, a neurological assessment was given to all macaques for detailed evaluation of neurological function. Briefly, evaluation included posture and gait assessment, cranial nerve assessment, proprioceptive assessment and spinal/nerve reflexes. At study day 56, macaques were euthanized, and gross postmortem examination and necropsy was performed. 25 major tissues were harvested from each macaque in duplicate for either snap freezing or fixation in formalin. DNA or RNA was purified from snap-frozen tissues and used for vector biodistribution or transgene expression analysis, respectively. For vector biodistribution, genome copies (gc) per total DNA weight were determined using a TaqMan qPCR assay with probes directed again the polyA region of the transgene cassette and an internal standard. To quantify transgene expression, total RNA was used to generate cDNA via first strand synthesis with polyT oligonucleotides, followed by TaqMan qPCR with probes specific for the transgene that did not cross-react with the endogenous rhesus MECP2 sequence. For histopathological analysis, macaque tissue was embedded into paraffin and these sections were stained with H&E solution, Myc-tag or MECP2 antibodies, respectively. Same spinal cord sections were incubated with Luxol Fast Blue to stain myelin. All stained tissues sections were reviewed by a board-certified veterinary pathologist, and abnormal findings verified by peer review.

2. AAVhu68.hSyn-MECP2

The AAVhu68.hSyn-MECP2 having the MECP coding sequence of SEQ ID NO: 3 (encoding hMECP of SEQ ID NO: 2) was tested in non-human primates. Rhesus macaques (age 5-7 years) were injected into the CSF via cisterna magna access (ICM) with different doses. No adverse signs were observed during the 56 days in-live phase and blood chemistry tests were all normal. After necropsy, vector biodistribution was determined by qPCR and a dose-dependent increase was found in all CNS tissues tested. This was mirrored in a dose-dependent increase of transgene mRNA in the same CNS tissues (determined by TaqMan qPCR with an assay selectively recognizing our transgene). Vector biodistribution was compared with transgene mRNA levels with NHP that had been administered via the same route of administration (ROA) a comparable dose of previously published vectors (Sinnett, S. E. et al. and Gadalla, K. K. E. et al., as cited herein). The AAV-hu68.hSyn-MECP2 vector showed a similar vector biodistribution compared to the other vectors, however, the vector was able to achieve much higher mRNA expression in all CNS tissues, which may make the vector better suited for efficacious Rett syndrome gene therapy.

Study 1. The following Rhesus Macaques population were studied.

| | Group | | Age | Weight |
|---|---|---|---|---|
| NHP ID | # | Gender | (years) | (kg) |
| RA3086 | 1 | F | 4.9 | 6.55 |
| RA3092 | 1 | F | 5.4 | 7.72 |
| RA1551 | 2 | M | 6.8 | 14.9 |
| RA3173 | 2 | M | 4.9 | 7.7 |
| RA3157 | 3 | M | 4.9 | 6.55 |
| RA3166 | 3 | M | 4.7 | 4.6 |
| RA2470 | 4 | M | 5.7 | 7.65 |
| RA3152 | 4 | M | 5.7 | 8.8 |

The AAV.hMECP2 vectors were produced using the AAVhu68 capsid and listed in the table below. Expression cassettes in the vectors combine different mouse Mecp2 promoter sequences with the wildtype human MECP2 coding sequence (noted as MECP2 or MECP2e1).

| NHP ID | Group | Vector |
|---|---|---|
| RA3086, | 1 | AAVhu68.MeP426.MECP2.RDH1.Stuffer |
| RA3092 | | |
| RA1551, | 2 | AAVhu68.MeP426.MECP2-myc.RDH1.Stuffer |
| RA3173 | | |
| RA3157, | 3 | AAVhu68sc.mMeP546.SVI.MeCP2e1.SpA |
| RA3166 | | (reference product) ) |
| RA2470, | 4 | AAVhu68.CB7.CI.MECP2.rBG |
| RA3152 | | |

The indicated vectors were delivered at a dose of 2×10¹³ GC/NHP via intra-cisterna magna (ICM) injection. The animals were then observed for 56 days after administration. Necropsy and pathology review were performed.

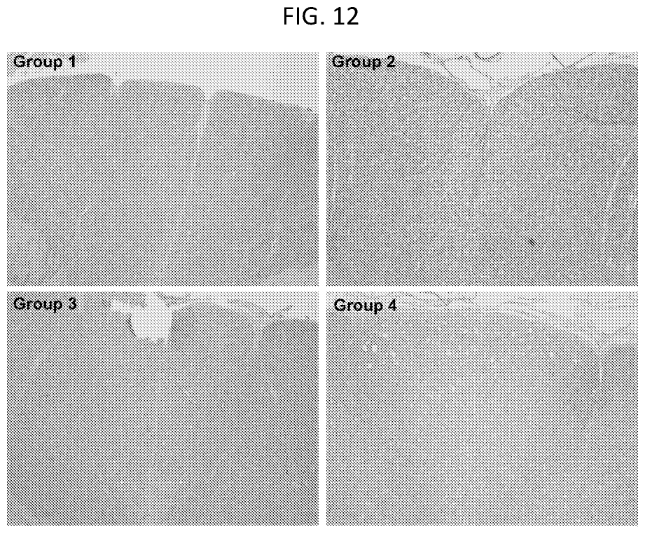

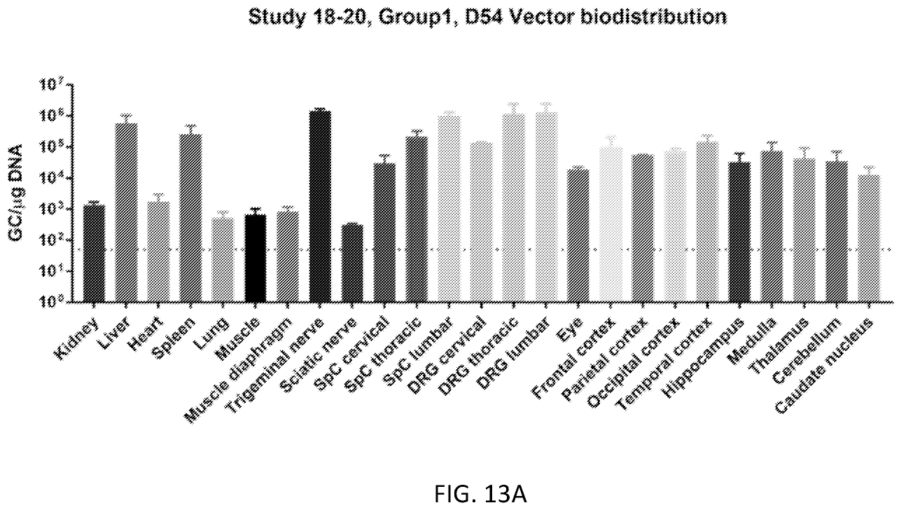

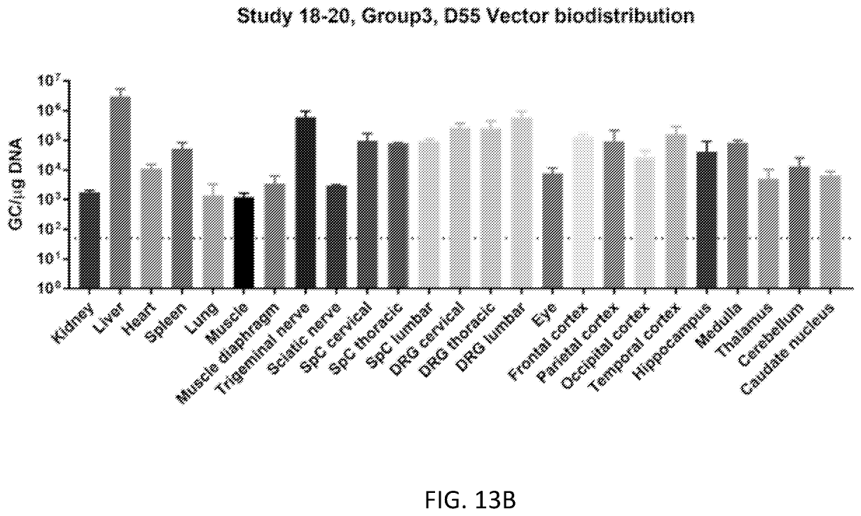

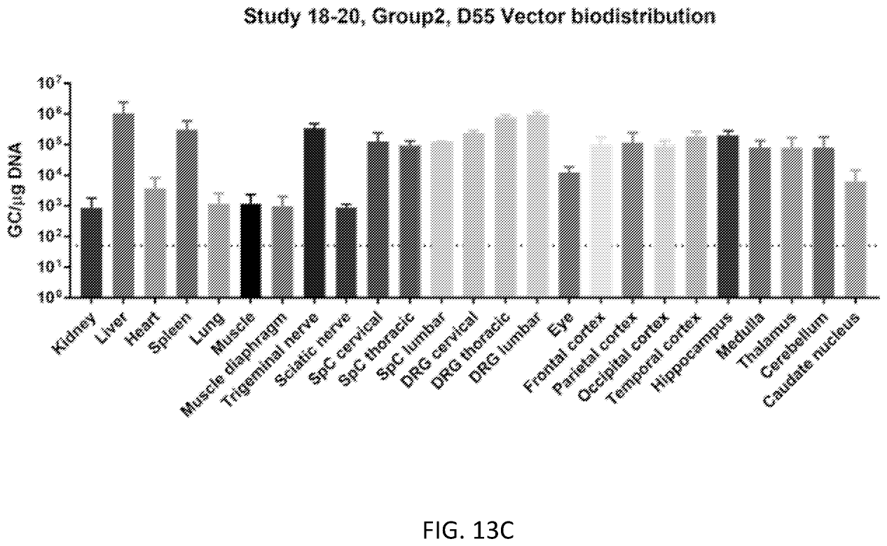

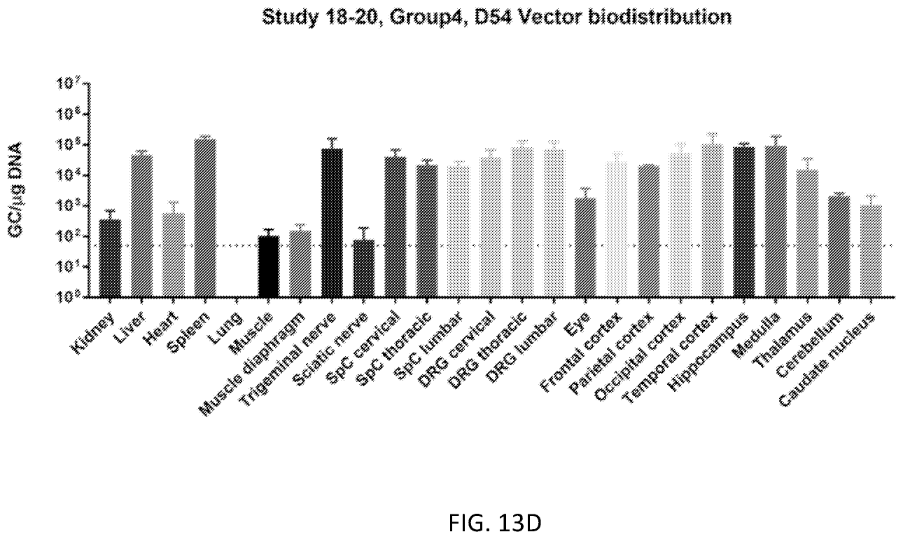

Axonopathy in dorsal white matter tracts characterized by dilated myelin sheaths with and without myelomacrophages and axonal fragmentation were investigated in Groups 1-4. As shown in FIG. 11, a minimal to moderate axonopathy was observed in Group 1 and 2, while a minimal to mild axonopathy was in Group 3 and a moderate to severe axonopathy in Group 4. Luxol Blue stain showed mild to moderate loss of myelin in Spinal White Matter Tracts of all tested groups. See, FIG. 12. FIGS. 13A to 13D revealed vector biodistribution across tissues.

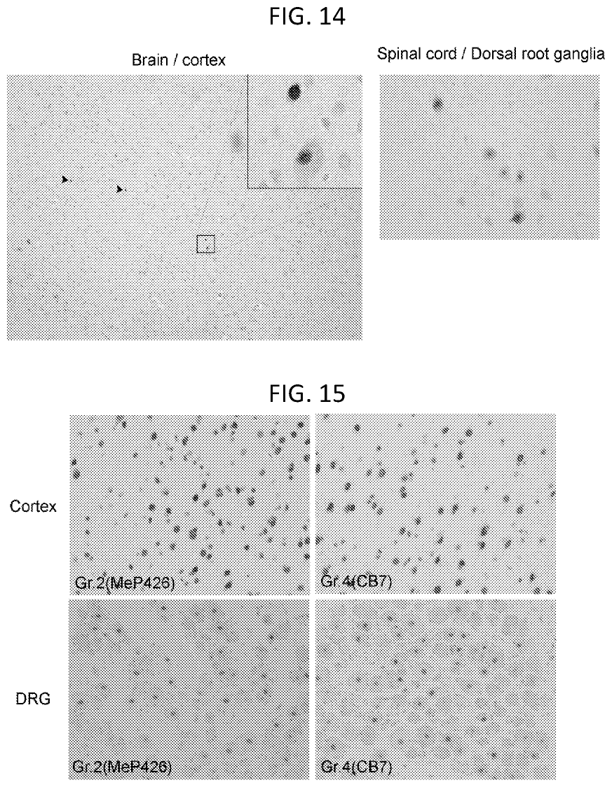

Further, MECP2 expression was accessed by IHC with myc antibodies in Group 2 (MeP426-MECP2_myc) or with MECP2 antibodies in Groups 2 and 4. See, FIGS. 14 and 15. Successful expression were detected in both brain cortex and dorsal root ganglia of spinal cord. hMECP2 mRNA expression was measured. Relative quantification with a RT-qPCR probe that selectively amplifies hMECP2 showed different expression levels per group and tissue though vector distribution was similar across groups. See the table below.

| Study | 18-20 | | | |
|---|---|---|---|---|
| Promoter | CB7 | mP426 | mP426 | mP546 |
| Transgene | hMECP2 | hMECP2 | hMECP2-myc | hMECP2 |
| dose | 2 × 10¹³ gc | 2 ×10¹³ gc | 2 × 10¹³ gc | 2 × 10¹³ gc |
| Temporal cortex | 100 | 9.3 | 2.2 | 17.4 |
| Frontal cortex | 81.1 | 1.9 | <1 | 7.2 |
| cerebellum | 19.3 | <1 | 1.6 | 12.2 |
| Liver/spleen | 136.1 | N/A | 317.6 | 355.7 |

4. Study 2

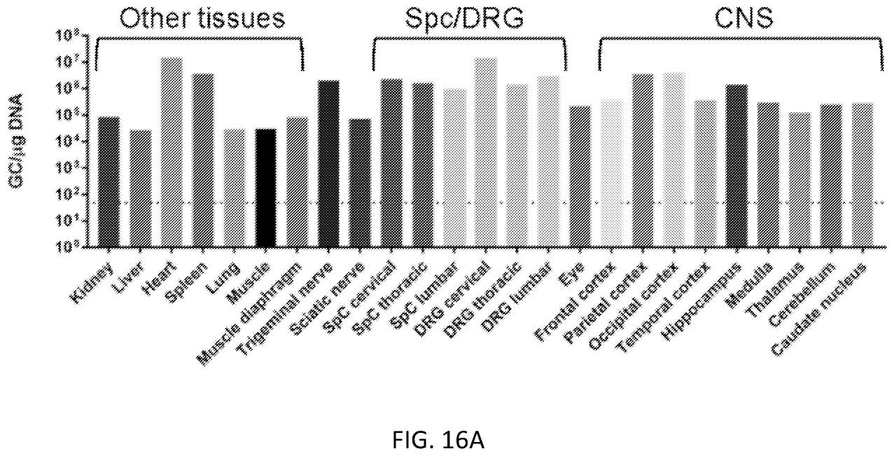

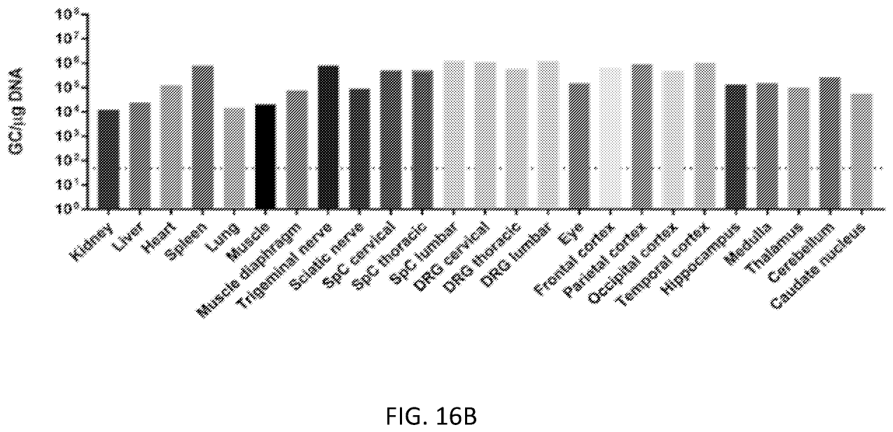

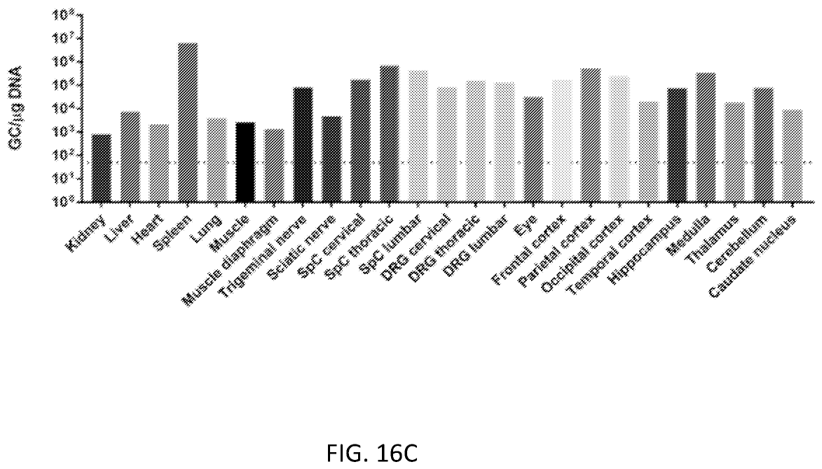

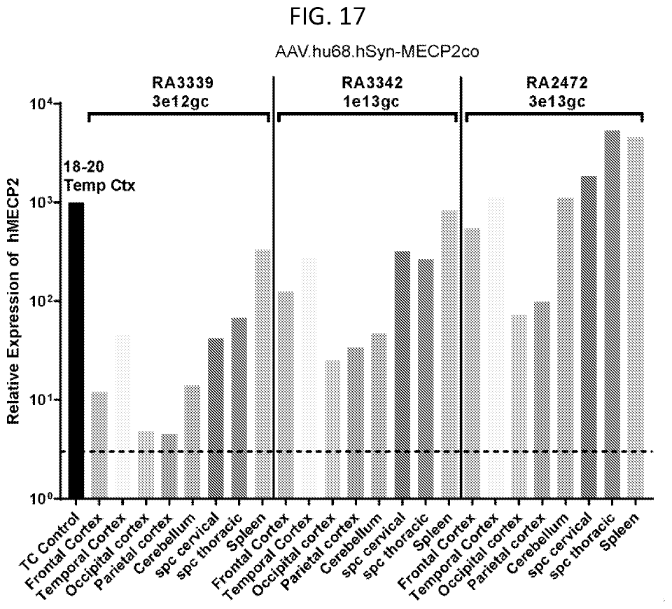

The AAVhu68.hSyn.MECP2co.SV40 vector comprising an engineered hMECP2 coding sequence driven by a hSyn promoter was produced and delivered to a NHP subject at 3×10¹³ GC/NHP, 1×10¹³ GC/NHP and 3×10¹² GC/NHP via ICM injection. Vector biodistribution using each dose was investigated and the result is shown in FIGS. 16A to 16C. Relative quantification with a RT-qPCR probe that selectively amplifies hMECP2 showed different expression levels per group and tissue (FIG. 17), though vector distribution was similar across groups. See, the table below.

| Study | 18-36 | | |
|---|---|---|---|
| Promoter | hSyn | | |
| Transgene | hMECP2_co (SEQ ID NO: 3) | | |
| Dose | 3 × 10¹² GC/NHP | 1 × 10¹³ GC/NHP | 3 × 10¹³ GC/NHP |
| Temporal cortex | 5 | 27 | 112 |
| Frontal cortex | 10 | 12 | 55 |
| cerebellum | 10 | 50 | 112 |
| Liver/spleen | 33 | 83 | 461 |

Expression profile for hSyn promoter is established and then compared to CBA promoter using GFP as readout.

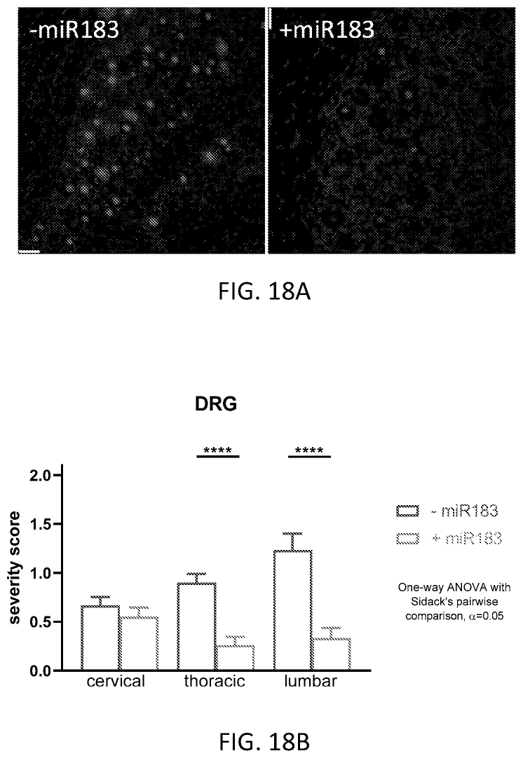

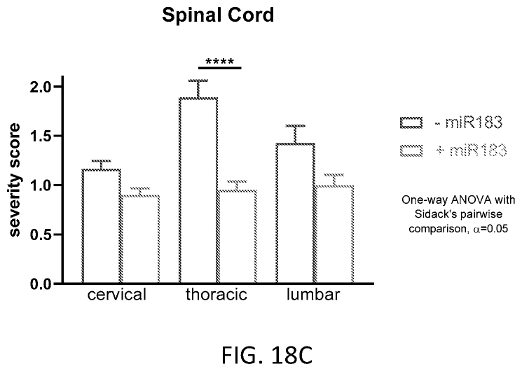

DRG de-targeting is incorporated in MECP2 expression cassette to allow for higher dose of the vector administrated. The AAVhu68.hSyn.MECP2co vector, comprising an engineered hMECP2 coding sequence driven by a hSyn promoter, or the AAVhu68.hSyn.MECP2co.miR183 (SEQ ID NO: 15), comprising an addition of miR183 sequences for DRG de-targeting, was delivered to a NHP subject at high dose of 1×10¹⁴ GC/NHP via ICM injection. Tissues were harvested at 3 months post-treatment from NHP subjects (N=3). In-situ hybridization with a probe specific to MECP2co showed that inclusion of the miR183 cassette drastically reduced the number of DRG cells that express MECP2co and the amount of RNA that is expressed (FIG. 18A). Additionally, pathologist scoring was performed of white matter tracks and DRG. For all tissue scores, there was a strong trend to or a significant decrease in severity in the NHP that had received the AAV vector with miR183 (FIG. 18B and FIG. 18C). Harvested tissues were also subjected to in situ hybridization assay, and quantified for DRG cell number and intensity.

### Table (Sequence Listing Free Text)

The following information is provided for sequences containing free text under numeric identifier <223>.

| SEQ ID NO: | |
|---|---|
| (containing free text) | Free text under <223 |
| 1 | <220> |
| | <221> repeat_region |
| | <222> (1) . . . (130) |
| | <223> ITR |
| | <220> |
| | <221> promoter |
| | <222> (213) . . . (678) |
| | <223> hSynapsin |
| | <220> |
| | <221> misc_feature |
| | <222> (690) . . . (695) |
| | <223> Kozak |
| | <220> |
| | <221> CDS |
| | <222> (696) . . . (2195) |
| | <223> human MeCP2alpha |
| | <220> |
| | <221> misc_feature |
| | <222> (2190) . . . (2195) |
| | <223> Stop codons |
| | <220> |
| | <221> polyA_signal |
| | <222> (2303) . . . (2534) |
| | <223> SV40 late polyadenylation |
| | <220> |
| | <221> repeat_region |
| | <222> (2599) . . . (2728) |
| | <223> ITR |
| | <220> |
| | <221> misc_feature |
| | <222> (2905) . . . (3343) |
| | <223> f1 ori |
| | <220> |
| | <221> misc_feature |
| | <222> (3372) . . . (4014) |
| | <223> pUC origin of replication |
| | <220> |
| | <221> misc_feature |
| | <222> (4689) . . . (5504) |
| | <223> Kan-r |
| | <220> |
| | <221> misc_feature |
| | <222> (5747) . . . (5932) |
| | <223> Lac promoter |
| 2 | <220> |
| | <223> Synthetic Construct |
| 3 | <220> |
| | <223> human MeCP2alpha |
| 4 | <220> |
| | <221> repeat_region |
| | <222> (1) . . . (130) |
| | <223> 5' ITR |
| | <220> |
| | <221> promoter |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> TATA_signal |
| | <222> (836) . . . (839) |
| | <223> TATA |
| | <220> |
| | <221> Intron |
| | <222> (957) . . . (1929) |
| | <223> chicken beta-actin intron |
| | <220> |
| | <221> misc_feature |
| | <222> (1993) . . . (3486) |
| | <223> MECP2 e1 |
| | <220> |
| | <221> misc_feature |
| | <222> (3490) . . . (3537) |
| | <223> SpA |
| | <220> |
| | <221> polyA_signal |
| | <222> (3604) . . . (3730) |
| | <223> Rabbit globin poly A |
| | <220> |
| | <221> repeat_region |
| | <222> (3819) . . . (3948) |
| | <223> 3' ITR |
| | <220> |
| | <221> rep_origin |
| | <222> (4125) . . . (4580) |
| | <223> fi ori |
| | <220> |
| | <221> misc_feature |
| | <222> (4711) . . . (5568) |
| | <223> AP(R) |
| | <220> |
| | <221> rep_origin |
| | <222> (5742) . . . (6330) |
| | <223> Origin of replication |
| 5 | <220> |
| | <223> MECP2 |
| 6 | <220> |
| | <221> repeat_region |
| | <222> (1) . . . (130) |
| | <223> ITR |
| | <220> |
| | <221> promoter |
| | <222> (213) . . . (678) |
| | <223> hSynapsin promoter |
| | <220> |
| | <221> misc_feature |
| | <222> (690) . . . (695) |
| | <223> Kozak |
| | <220> |
| | <221> misc_feature |
| | <222> (696) . . . (2195) |
| | <223> human MeCP2 coding sequence |
| | <220> |
| | <221> misc_feature |
| | <222> (2190) . . . (2195) |
| | <223> stop codons |
| | <220> |
| | <221> misc_feature |
| | <222> (2256) . . . (2277) |
| | <223> miRNA183 |
| | <220> |
| | <221> misc_feature |
| | <222> (2282) . . . (2303) |
| | <223> miRNA183 |
| | <220> |
| | <221> misc_feature |
| | <222> (2310) . . . (2331) |
| | <223> miRNA183 |
| | <220> |
| | <221> misc_feature |
| | <222> (2338) . . . (2359) |
| | <223> miRNA183 |
| | <220> |
| | <221> polyA_signal |
| | <222> (2377) . . . (2608) |
| | <223> SV40 late polyadenylation signal |
| | <220> |
| | <221> repeat_region |
| | <222> (2673) . . . (2802) |
| | <223> ITR |
| | <220> |
| | <221> misc_feature |
| | <222> (2979) . . . (3417) |
| | <223> f1 ori |
| | <220> |
| | <221> misc_feature |
| | <222> (3446) . . . (4088) |
| | <223> pUC origin of replication |
| | <220> |
| | <221> misc_feature |
| | <222> (4763) . . . (5578) |
| | <223> Kan-r |
| | <220> |
| | <221> misc_feature |
| | <222> (5821) . . . (6006) |
| | <223> Lac promoter |
| 7 | <220> |
| | <223> miR183 binding site |
| 8 | <220> |
| | <223> hMECP2 Isoform A |
| 9 | <220> |
| | <223> miRNA target sequence. |
| 10 | <220> |
| | <223> miRNA target sequence. |
| 11 | <220> |
| | <223> miRNA target sequence. |
| 12 | <220> |
| | <223> chicken beta actin promoter |
| | with a cytomegalovirus enhancer (CB7) |
| 13 | <220> |
| | <223> human elongation initiation |
| | factor 1 alpha promoter (EF1a) |
| 14 | <220> |
| | <223> spacer |
| 15 | <220> |
| | <223> expression vector |
| | <220> |
| | <221> repeat_region |
| | <222> (1) . . . (130) |
| | <223> 5'ITR |
| | <220> |
| | <221> promoter |
| | <222> (213) . . . (678) |
| | <223> hSynapsin |
| | <220> |
| | <221> misc_feature |
| | <222> (690) . . . (695) |
| | <223> Kozak |
| | <220> |
| | <221> misc_feature |
| | <222> (696) . . . (2195) |
| | <223> human Mecp2alpha |
| | <220> |
| | <221> misc_binding |
| | <222> (2256) . . . (2359) |
| | <223> (4x) |
| | <220> |
| | <221> polyA_signal |
| | <222> (2377) . . . (2608) |
| | <223> SV40 late polyadenylation signal |
| | <220> |
| | <221> repeat_region |
| | <222> (2673) . . . (2802) |
| | <223> 3'ITR |
| 16 | <220> |
| | <223> mRNA transcript variant |
| 17 | <220> |
| | <223> mRNA transcript variant |
| 18 | <220> |
| | <223> mRNA transcript variant |

All documents cited in this specification are incorporated herein by reference, as in priority application, U.S. Patent Application No. 62/837,947, filed Apr. 24, 2019. The Sequence Listing filed herewith, labelled “19-9007PCT_SeqListing_ST25.txt”, and the sequences and text therein are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) useful for treating Rett Syndrome, wherein the rAAV comprises:
(a) an adeno-associated virus (AAV) capsid; and
(b) a vector genome packaged in the AAV capsid of (a), wherein the vector genome comprises inverted terminal repeats (ITR) and a nucleic acid sequence encoding a functional human methyl-CpG binding protein 2 (hMECP2) under control of regulatory sequences which direct the hMECP2 expression in central nervous system cells, and wherein the hMECP2-coding sequence is SEQ ID NO: 3 or a sequence at least about 95% identical to SEQ ID NO: 3.

2. The rAAV according to claim 1, wherein the hMECP-coding sequence is less than 80% identical to any one of hMECP transcript variants 1 to 3 (NM_001316337.1 (SEQ ID NO: 16), NM_004992.3 (SEQ ID NO: 17) and NM_001110792.1 (SEQ ID NO: 18)).

3. The rAAV according to claim 1, wherein the functional hMECP2 has an amino acid sequence of SEQ ID NO: 2.

4. The rAAV according to claim 1, wherein the regulatory sequences comprise a neuron specific promoter.

5. The rAAV according to claim 1, wherein the regulatory sequences comprise a constitutive promoter.

6. The rAAV according to claim 1, wherein the regulatory sequences comprise a human Synaspin promoter or a CB7 promoter.

7. The rAAV according to claim 1, wherein the regulatory sequences further comprise one or more of a Kozak sequence, a polyadenylation sequence, an intron sequence, an enhancer sequence, a TATA signal sequence and a polyA signal sequence.

8. The rAAV according to claim 1, wherein the vector genome further comprises at least two tandem repeats of dorsal root ganglion (drg)-specific miRNA target sequences in the 3′ untranslated region for the hMECP, wherein the at least two tandem repeats comprise at least a first miRNA target sequence and at least a second miRNA target sequence which may be the same or different, and target miR183 or miR182.

9. The rAAV according to claim 1, wherein the miRNA target sequence for the at least first and/or the at least second miRNA target sequence for the expression cassette mRNA or DNA positive strand is (i) AGTGAATTCTACCAGTGCCATA (miR183, SEQ ID NO: 7); and (ii) AGCAAAAATGTGCTAGTGCCAAA (SEQ ID NO: 9).

10. The rAAV according to claim 8, wherein the two or more of the miRNA target sequences are separated by a spacer and each spacer is independently selected from one or more of (A) GGAT; (B) CACGTG; or (C) GCATGC.

11. The rAAV according to claim 10, wherein the spacer located between the miRNA target sequences may be located 3′ to the first miRNA target sequence and/or 5′ to the last miRNA target sequence.

12. The rAAV according to claim 10, wherein the spacers between the miRNA target sequences are the same.

13. The rAAV according to claim 1, wherein the vector genome comprises nucleic acids 1 to 2802 of SEQ ID NO: 6.

14. The rAAV according to claim 1, wherein the AAV capsid is an AAVhu68 capsid.

15. A composition comprising a stock of rAAV according to claim 1 and an aqueous suspension media.

16. The composition according to claim 15, wherein the suspension is formulated for intravenous delivery, intrathecal administration, or intracerebroventricular administration.

17. A vector comprising an expression cassette, wherein the expression cassette comprises a nucleic acid sequence encoding a functional human methyl-CpG binding protein 2 (hMECP2) under control of regulatory sequences which direct the hMECP2 expression, and wherein the hMECP2-coding sequence is SEQ ID NO: 3 or a sequence at least about 95% identical to SEQ ID NO: 3.

18. The vector according to claim 17, wherein the vector is a viral vector selected from a recombinant parvovirus, a recombinant lentivirus, a recombinant retrovirus, or a recombinant adenovirus; or a non-viral vector selected from a naked DNA, a naked RNA, an inorganic particle, a lipid particle, a polymer-based vector, or a chitosan-based formulation.

19. A method of treating Rett Syndrome, comprising administrating an effective amount of the rAAV according to claim 1 to a subject in need thereof.

20. An rAAV production system useful for producing the rAAV according to claim 1, wherein the production system comprises a cell culture comprising:
(a) a nucleic acid sequence encoding a Clade F AAV capsid protein;
(b) a vector genome; and
(c) a sufficient AAV rep functions and helper functions to permit packaging of the vector genome into the Clade F AAV capsid.

21. The rAAV production system according to claim 20, wherein the vector genome is nt 1 to nt 2728 of SEQ ID NO: 1, or nt 1 to nt 3949 of SEQ ID NO: 4, or nt 1 to nt 2802 of SEQ ID NO: 6.

22. The rAAV production system according to claim 20, wherein the cell culture is a human embryonic kidney 293 cell culture.

23. The rAAV production system according to claim 20, wherein the AAV rep is from a different AAV.

24. The rAAV production system according to claim 23, wherein the AAV rep is from AAV2.

25. The rAVV production system according to claim 20, wherein the AAV rep coding sequence and cap genes are on the same nucleic acid molecule, wherein there is optionally a spacer between the rep sequence and cap gene.

26. The rAAV production system according to claim 25, wherein the spacer is atgacttaaaccaggt (SEQ ID NO: 14).
